(19) United States
(12) Patent Application Publication
(10) Pub. No.: US 2025/0143862 A1
(43) Pub. Date: May 8, 2025

(54) METHODS AND DEVICES FOR PRE-VASCULARIZATION OF IMPLANTATION SITES AND/OR CELL ENCAPSULATION DEVICES

(71) Applicant: Encellin, Inc., Palo Alto, CA (US)

(72) Inventors: Crystal NYITRAY, San Francisco, CA (US); Grace WEI, Tiburon, CA (US)

(21) Appl. No.: 18/735,879

(22) Filed: Jun. 6, 2024

Related U.S. Application Data
(63) Continuation of application No. PCT/US2022/052413, filed on Dec. 9, 2022.
(60) Provisional application No. 63/423,925, filed on Nov. 9, 2022.
(60) Provisional application No. 63/287,826, filed on Dec. 9, 2021.

(51) Int. Cl. A61F 2/02; A61F 2/00; A61L 27/34; A61L 27/56; C08L 67/04

(52) U.S. Cl. CPC A61F 2/022; A61L 27/34; A61L 27/56; C08L 67/04; A61F 2002/0086; A61F 2210/0076

(57) ABSTRACT

Provided herein are devices and methods for improved oxygenation of biological cells transplanted into a biological subject. The devices and methods may be used to promote vascularization to a transplantation site of the biological subject, and/or may be used to improve survival and functionality of cells contained within an encapsulation device.

### CROSS-REFERENCE

This application is a continuation of International Application No. PCT/US2022/052413 filed Dec. 9, 2022, which claims the benefit of U.S. Provisional Application No. 63/287,826, filed Dec. 9, 2021, and U.S. Provisional Application No. 63/423,925, filed Nov. 9, 2022, all of which are incorporated herein by reference in their entirety.

### BACKGROUND

The transplantation of cells into a subject to produce therapeutic molecules may be an attractive alternative to the use of exogenous drugs. The encapsulation of cell transplants may be required to ensure cell localization by containing the transplanted cells. The encapsulation of cell transplants may further be required to help protect the cell transplants from immune attack and to prevent their proliferation or migration in the host. Additionally, transplanted cells may require an adequate oxygen supply to improve their survival rates in the subject. Cell containment devices with features such as the ability to be prevascularized or release vascularizing or enhancing factors may improve their survival rates in the subject. The present disclosure provides methods and devices for improved survival of encapsulation devices and therapeutic cells contained within. The present disclosure further provides methods and devices for improved survival of encapsulated cells.

### SUMMARY

In one aspect, a method is provided comprising: a) exposing a plurality of cells to low oxygen conditions thereby generating a plurality of pre-conditioned cells; and b) implanting an encapsulation device comprising the plurality of pre-conditioned cells into an implantation site of a subject. In some cases, the exposing of a) comprises exposing the plurality of cells to low oxygen conditions in a cyclical fashion. In some cases, the exposing of a) comprises exposing the plurality of cells to low oxygen conditions in a continuous fashion. In some cases, the exposing of a) comprises incubating the plurality of cells in low oxygen conditions for at least one hour to at least one week prior to the implanting of b). In some case, the low oxygen conditions comprise from about 0.1% to about 5.0% oxygen. In some cases, the plurality of cells are loaded into the device prior to the exposing of a). In some cases, the plurality of pre-conditioned cells are loaded into the device prior to the implanting of b).

In another aspect, a method is provided comprising: a) implanting a pre-vascularization device into an implantation site of a subject: b) removing the pre-vascularization device from the implantation site of the subject; and c) implanting an encapsulation device into the implantation site of the subject. In some cases, the pre-vascularization device and the encapsulation device are of substantially similar dimensions. In some cases, the pre-vascularization device and the encapsulation device are of a substantially similar shape. In some cases, the pre-vascularization device and the encapsulation device are composed of a similar material. In some cases, the implanting of a) comprises implanting the pre-vascularization device into the implantation site of the subject for at least one week prior to the removing of b). In some cases, the encapsulation device comprises a plurality of cells. In some cases, the plurality of cells is contained within a lumen of the encapsulation device. In some cases, the encapsulation device comprises a semi-permeable membrane. In some cases, the pre-vascularization device comprises an impermeable membrane. In some cases, the impermeable membrane comprises poly-caprolactone (PCL).

In another aspect, a method is provided comprising: a) implanting a pre-vascularization device into an implantation site of a subject; and b) implanting an encapsulation device into the implantation site of the subject. In some cases, the pre-vascularization device and the encapsulation device are of similar dimensions. In some cases, the pre-vascularization device and the encapsulation device are of a similar shape. In some cases, the pre-vascularization device and the encapsulation device are composed of a similar material. In some cases, the encapsulation device comprises a plurality of cells. In some cases, the plurality of cells is contained within a lumen of the encapsulation device. In some cases, the encapsulation device comprises a semi-permeable membrane. In some cases, the pre-vascularization device comprises an impermeable membrane. In some cases, the impermeable membrane comprises poly-caprolactone (PCL).

In another aspect, a method is provided comprising: a) implanting an encapsulation device into an implantation site of a subject for a defined period of time, wherein the encapsulation device does not comprise cells; and b) loading the encapsulation device with a plurality of cells after the defined period of time. In some cases, after the defined period of time, at least 10% of a surface of the encapsulation device is vascularized. In some cases, the defined period of time is from about 2 to about 3 weeks.

In another aspect, a method is provided comprising: a) introducing an oxygenation device to an implantation site of a subject for a defined period of time, wherein the oxygenation device is configured to deliver oxygen to the implantation site; and b) implanting an encapsulation device into the implantation site, wherein the encapsulation device comprises a plurality of cells contained within a lumen of the encapsulation device. In some cases, the introducing of a) occurs prior to the implanting of b). In some cases, the introducing of a) occurs at the same time as the implanting of b). In some cases, the oxygenation device is rechargeable in situ. In some cases, the oxygenation device is enclosed within a semi-permeable membrane.

In another aspect, a method is provided comprising: (i) introducing one or more chemical supplements to an implantation site of a subject and (ii) introducing an encapsulation device to the implantation site, wherein the one or more chemical supplements provide controlled release of oxygen to the implantation site. In some cases, the encapsulation device comprises a plurality of cells enclosed within a lumen of the encapsulation device. In some cases, the introducing the one or more chemical supplements to the implantation site occurs substantially at the same time as the introducing the encapsulation device to the implantation site. In some cases, the introducing the one or more chemical supplements to the implantation site occurs prior to the introducing the encapsulation device to the implantation site. In some cases, the one or more chemical supplements are contained within the encapsulation device. In some cases, the one or more chemical supplements are selected from the group consisting of: polyethyleneglycole diacrylate (PEGDA), HEMOXCell, PolyHeme®, HemoLink™, Hemopure™, perfluorodecalin (PFD), copper(I) perfluorate, calcium perfluorate, aluminum perfluorate, ammonium perchlorate, perchloric acid, potassium perchlorate, sodium perchlorate, perfluorohexyloctane, zinc, manganese, ruthenium, and iron. In some cases, the one or more chemical supplements are selected from the group comprising: VEGF (vascular endothelial growth factor), PDGF (platelet-derived growth factor). FGF-1 (fibroblast growth factor), angiopoietin MCP-1, avf33, avf35, CD-31, VE-cadherin, ephrin, plasminogen activators, angiogenin. Del-1, aFGF (acid fibroblast growth factor), vFGF (basic fibroblast growth factor), follistatin, G-CSF (granulocyte colony-stimulating factor). HGF (hepatocyte growth factor). Leptin, placental growth factor. PD-ECGF (platelet-derived endothelial growth factor), or combinations thereof.

In another aspect, a method is provided comprising introducing an encapsulation device to an implantation site of a subject, wherein the encapsulation device comprises: (i) a plurality of therapeutic cells contained within a lumen of the encapsulation device; and (ii) a plurality of supporting cells contained with the lumen of the encapsulation device, wherein the plurality of supporting cells promote oxygenation of the implantation site. In some cases, the plurality of supporting cells promote survival of transplanted cells. In some cases, the plurality of supporting cells promote engraftment. In some cases, the plurality of supporting cells are selected from the group consisting of: endothelial cells, blood cells, heme-containing cells, heme-like oxygen chelating cells, thyroid cells, parathyroid cells, bone marrow cells, mesenchymal stem cells, stromal cells, pluripotent stem cells, induced pluripotent stem cells, embryonic stem cells, blood vessel cells, precursor cells derived from adipose tissue or from bone marrow derived progenitor cells, or from intestinal cells, islets or islet cells. Sertoli cells, beta cells, progenitors of islet cells, progenitors of beta cells, peripheral blood progenitor cells, stem cells or their derivatives and/or cells/stem cells isolated from adult tissue, retinal progenitor/derivative cells, cardiac progenitor/derivative cells, osteoprogenitor cells or derivatives thereof, neuronal progenitor cells or derivatives thereof, and any combination thereof.

In some cases of the method of any one of the preceding aspects, the encapsulation device comprises: (a) a lumen for enclosing a plurality of cells: (b) a biocompatible polymer surrounding the lumen and comprising a plurality of pores, wherein, when the lumen comprises insulin-secreting cells, the insulin-secreting cells maintain a glucose stimulation index (GSI) for at least 20 days in vitro of at least 75% of a GSI as measured from Day 1 by a glucose stimulation assay, and wherein the device has a thickness of less than 40 μm. In some cases, the plurality of pores are micropores having an average diameter of less than 5 μm. In some cases, the plurality of pores are micropores having an average diameter of greater than 1 μm.

In some cases of the method of any one of the preceding aspects, the encapsulation device comprises: (a) a lumen for enclosing a plurality of cells: (b) a biocompatible polymer surrounding the lumen and comprising a plurality of pores, wherein, when the lumen comprises insulin-secreting cells, the insulin-secreting cells maintain a glucose stimulation index (GSI) for at least 20 days in vitro of at least 75% of a GSI as measured from Day 1 by a glucose stimulation assay, and wherein the plurality of pores have a connectivity diameter from 100 nm to 900 nm. In some cases, the glucose stimulation assay comprises: (i) incubating the insulin-secreting cells in 5 mM glucose for 30 minutes; (ii) incubating the insulin-secreting cells in 15 mM glucose for 30 minutes; and (iii) measuring a level of insulin secreted from the insulin-secreting cells in step (i) and step (ii). In some cases, the GSI is a ratio of the level of insulin secreted from the insulin-secreting cells in step (i) to the level of insulin secreted from the insulin-secreting cells in step (ii). In some cases, the insulin-secreting cells are MIN6 cells. In some cases, the plurality of cells, when present in the method, are present in an effective number for treatment of a subject in need thereof. In some cases, the biocompatible polymer comprises a first layer of biocompatible polymer and a second layer of biocompatible polymer, wherein the lumen is disposed between the first layer of biocompatible polymer and the second layer of biocompatible polymer. In some cases, the first layer of biocompatible polymer and the second layer of biocompatible polymer comprise the same biocompatible polymer. In some cases, the first layer of biocompatible polymer and the second layer of biocompatible polymer are each less than 20 μm in thickness. In some cases, the plurality of pores are nanopores having an average diameter from 5 nm to 500 nm. In some cases, the plurality of pores have an average diameter from 10 nm to 300 nm. In some cases, the biocompatible polymer is selected from the group comprising: methacrylate polymer, polyethyleneimine, polyethyleneimine-dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapatite, poly(lactic acid), polyhydroxyvalerte and copolymers thereof, polyhydroxy butyrate and copolymers thereof, polycaprolactone, polydiaxanone, polyanhydride, polycyanocrylate, poly(amino acids), poly(orthoesters), polyesters, collagen, gelatin, cellulose polymer, chitosans, alginates, and any combination thereof. In some cases, the biocompatible polymer comprises polycaprolactone. In some cases, the device is configured for implantation into a subject. In some cases, the implantation is selected from the group consisting of: subcutaneous, omentum, intraperitoneal, and intramuscular implantation. In some cases, the subject is a mammal. In some cases, the subject is a human. In some cases, the plurality of cells are selected from the group consisting of: bone marrow cells, mesenchymal stem cells, stromal cells, pluripotent stem cells, induced pluripotent stem cells, embryonic stem cells, blood vessel cells, precursor cells derived from adipose tissue, bone marrow derived progenitor cells, intestinal cells, islets. Sertoli cells, beta cells, progenitors of islet cells, progenitors of beta cells, peripheral blood progenitor cells, stem cells isolated from adult tissue, retinal progenitor cells, cardiac progenitor cells, osteoprogenitor cells, neuronal progenitor cells, genetically transformed cells, and any combination thereof. In some cases, the plurality of cells are autologous cells, allogeneic cells, xenogeneic cells, or any combination thereof. In some cases, the plurality of cells are insulin-secreting cells. In some cases, the device is used to treat a subject in need thereof. In some cases, the subject in need thereof has or is suspected of having diabetes mellitus. In some cases, the device is used to transplant the plurality of cells into a subject, and wherein the plurality of cells are introduced into the lumen after the device has been implanted into the subject. In some cases, the device is used to transplant the plurality of cells into a subject, and wherein the plurality of cells are introduced into the lumen before the device has been implanted into the subject. In some cases, the device is configured to allow release of a therapeutic protein secreted by the plurality of cells from the lumen of the device. In some cases, the biocompatible polymer substantially prevents ingress of immune cells and immunoglobulins into the lumen. In some cases, the plurality of pores have a connectivity diameter from 100 nm to 900 nm. In some cases, the device comprises a thickness of less than 40 μm.

In some cases of the method of any one of the preceding aspects, the encapsulation device comprises: a) a first polymer layer comprising a plurality of pores having an average connectivity diameter of less than about 500 nm; and b) a second polymer layer comprising a plurality of pores having an average connectivity diameter of greater than about 100 μm: wherein the first polymer layer and the second polymer layer define a lumen for enclosing a plurality of cells. In some cases, the plurality of pores present in the first polymer layer have an average connectivity diameter from about 10 nm to about 200 nm. In some cases, the plurality of pores present in the first polymer layer have an average connectivity diameter from about 180 nm to about 220 nm. In some cases, the plurality of pores present in the second polymer layer have an average connectivity diameter from about 100 μm to about 500 μm. In some cases, the plurality of pores present in the second polymer layer have an average connectivity diameter from about 200 μm to about 400 μm. In some cases, the plurality of pores present in the first polymer layer have an average pore diameter from about 1 μm to about 5 μm. In some cases, the plurality of pores present in the second polymer layer have an average pore diameter from about 500 μm to about 3 mm. In some cases, the plurality of pores present in the second polymer layer have an average pore diameter from about 100 μm to about 1 mm. In some cases, the first polymer layer has a thickness from about 10 μm to about 200 μm. In some cases, the first polymer layer has a thickness from about 20 μm to about 50 μm. In some cases, the first polymer layer has a thickness from about 18 μm to about 22 μm. In some cases, the second polymer layer has a thickness from about 1 mm to about 5 mm. In some cases, the second polymer layer has a thickness from about 400 μm to about 600 μm. In some cases, the, further comprising a third polymer layer having an average connectivity diameter of less than about 500 nm. In some cases, further comprising a port for introducing the plurality of cells into the lumen of the device. In some cases, the first polymer layer, the second polymer, the third polymer layer, or any combination thereof, comprises a biocompatible polymer selected from the group consisting of: polycaprolactone (PCL), polyvinylidene fluoride (PVDF), polytetrafluoroethylene (PTFE), polyethylene (PE), methacrylate polymer, polyethyleneimine, polyethyleneimine-dextran sulfate, poly(vinylsiloxane) ecopolymerepolyethyleneimine, phosphorylcholine, poly(ethyl methacrylate), polyurethane, poly(ethylene glycol) (PEG), poly(lactic-glycolic acid) (PLGA), hydroxyapatite, poly(lactic acid), polyhydroxyvalerte and copolymers thereof, polyhydroxy butyrate and copolymers thereof, polydiaxanone, polyanhydride, polycyanocrylate, poly(amino acids), poly(orthoesters), polyesters, collagen, gelatin, cellulose polymer, chitosans, alginates, laminin, and any combination thereof. In some cases, the biocompatible polymer is polycaprolactone. In some cases, the plurality of cells are selected from the group consisting of: thyroid cells, parathyroid cells, bone marrow cells, mesenchymal stem cells, stromal cells, pluripotent stem cells, induced pluripotent stem cells, embryonic stem cells, blood vessel cells, cells derived from adipose tissue, cells derived from bone marrow, intestinal cells or cells derived therefrom, islets or islet cells. Sertoli cells, beta cells, progenitors of islet cells, progenitors of beta cells, peripheral blood progenitor cells, stem cells or derivatives thereof isolated from adult tissue, retinal progenitor derivative cells, cardiac progenitor derivative cells, osteoprogenitor cells, neuronal progenitor cells, genetically transformed cells, and any combination thereof. In some cases, the first polymer layer and the second polymer layer are directly sealed along a periphery of the first polymer layer and the second polymer layer. In some cases, the encapsulation device does not comprise a support or frame. In some cases, the first polymer layer, the second polymer layer, or both, are non-laminated polymer layers.

In another aspect, a method is provided comprising (i) generating oxygen gas through an oxygen generator and (ii) delivering the oxygen gas to a cell encapsulation device, wherein the oxygen gas is delivered from the oxygen generator to the cell encapsulation device through a connection port. In some cases, the connection port connects the cell encapsulation device and the oxygen generator prior to implantation of cells to the cell encapsulation device. In some cases, the cell encapsulation device comprises a built-in port, wherein the built-in port allows for delivery of cells into a lumen of the cell encapsulation device. In some cases, the lumen comprises an oxygen delivery pathway. In some cases, the oxygen delivery pathway comprises multiple channels within the cell encapsulation device. In some cases, the cell encapsulation device comprises one or more sensors to detect oxygen levels in a subject, wherein the one or more sensors activate the oxygen generator to produce and deliver oxygen. In some cases, the one or more sensors are used to detect other chemicals, wherein the other chemicals comprise glucose. In some cases, the oxygen generator produces and delivers oxygen by electrophoresis or by chemical generation. In some cases, the oxygen generator in implanted in a subject at an implantation site. In some cases, the oxygen generator delivers oxygen to the cells contained within the cell encapsulation device and surrounding tissues of the implantation site

In another aspect, a device is provided comprising polycaprolactone (PCL) polymer, wherein the exterior surface is configured to be in contact with a bodily fluid or a bodily tissue of a subject, and wherein, when the device is implanted in a subject for at least 5 months, a level of fibrosis around the device has a fibrosis score of Grade 2 or less, as measured by histology, and wherein the PCL polymer has a number average molecular weight of 50-200 kDa. In some cases, the exterior surface is coated with a molecule that promotes vascularization of the device, a molecule that inhibits an immune response to the device, a molecule that inhibits an inflammatory response to the device, or any combination thereof. In some cases, the exterior surface is coated with one or more molecules selected from the group consisting of: vascular endothelial growth factor (VEGF), platelet-derived growth factor (PDGF), fibroblast growth factor (FGF-1), angiopoietin, monocyte chemoattractant protein-1 (MCP-1), alphaᵥbeta₃, alphaᵥbeta₃. CD-31. VE-cadherin, ephrin, plasminogen activators, angiogenin. Del-1, acid fibroblast growth factor (aFGF), basic fibroblast growth factor (bFGF), follistatin, granulocyte colony-stimulating factor (G-CSF), hepatocyte growth factor (HGF), leptin, placental growth factor, platelet-derived endothelial growth factor (PD-ECGF), and any combination thereof. In some cases, the device further comprises a lumen. In some cases, the lumen is configured to encapsulate a plurality of cells, a plurality of therapeutic molecules, or both. In some cases, the device is configured such that the plurality of therapeutic molecules are capable of diffusing out of the lumen, out of the device, or both. In some cases, the plurality of cells are selected from the group consisting of: bone marrow cells, mesenchymal stem cells, stromal cells, pluripotent stem cells, induced pluripotent stem cells, embryonic stem cells, blood vessel cells, precursor cells derived from adipose tissue, bone marrow derived progenitor cells, intestinal cells, islets, Sertoli cells, beta cells, progenitors of islet cells, progenitors of beta cells, peripheral blood progenitor cells, stem cells isolated from adult tissue, retinal progenitor cells, cardiac progenitor cells, osteoprogenitor cells, neuronal progenitor cells, genetically transformed cells, and any combination thereof. In some cases, the device further comprises a first layer of biocompatible polymer and a second layer of biocompatible polymer. In some cases, the first layer of biocompatible polymer, the second layer of biocompatible polymer, or both, comprise a plurality of nanopores. In some cases, the first layer of biocompatible polymer, the second layer of biocompatible polymer, or both, are selected from the group consisting of: methacrylate polymer, polyethyleneimine, polyethyleneimine-dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapatite, poly(lactic acid), polyhydroxyvalerte and copolymers thereof, polyhydroxy butyrate and copolymers thereof, polycaprolactone, polydiavanone, polyanhydride, polycyanocrylate, poly(amino acids), poly(orthoesters), polyesters, collagen, gelatin, cellulose polymer, chitosans, alginates, and any combination thereof. In some cases, the device is configured to be implanted into the subject. In some cases, the device is configured to be implanted into the subject by subcutaneous implantation, omentum implantation, intraperitoneal implantation, or intramuscular implantation. In some cases, the device is configured to be implanted into brain, spinal cord, pancreas, liver, uterus, skin, bladder, kidney, or muscle of the subject. In some cases, the subject is selected from the group consisting of: a human, a monkey, a dog, a cat, a cow, a horse, a pig, a sheep, a goat, a bear, a panda, a lion, a tiger, a leopard, an elephant, a camel, a zebra, a giraffe, a gorilla, a dolphin, and a whale.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts non-limiting examples of systems as provided herein.

FIG. 2 depicts non-limiting examples of systems as provided herein.

FIG. 3 depicts images illustrating that enhancement factors may be delivered inside, near, or to an Encellin cell encapsulation device (ENC-CED) to provide a benefit.

FIG. 4 depicts images illustrating that oxygen may be delivered inside, near, or to an ENC-CED to enhance oxygen in the ENC-CED.

FIG. 5 depicts images illustrating that factors may be delivered inside, near, or to an ENC-CED to enhance factors in the ENC-CED.

FIG. 6 depicts images illustrating that the ENC-CED may be attached to an implantation site in a subject.

### DETAILED DESCRIPTION

Disclosed herein are oxygenation devices and methods for improved oxygenation of cell transplantation sites. Further disclosed herein are oxygenation devices and methods for improved oxygenation of cell transplantation devices. The oxygenation devices and methods disclosed here may improve the survival and functionality of transplanted cells into a subject host (e.g., a human). The devices and methods disclosed herein may further improve engraftment and/or vascularization. The devices and methods disclosed herein may further improve survival rates and functionality of therapeutic cells encapsulated within an encapsulation device described here.

Disclosed herein are methods comprising: a) exposing a plurality of cells to low oxygen conditions thereby generating a plurality of pre-conditioned cells; and b) implanting an encapsulation device comprising said plurality of pre-conditioned cells into an implantation site of a subject. Further disclosed herein are methods comprising: a) implanting a pre-vascularization device into an implantation site of a subject: b) removing said pre-vascularization device from said implantation site of said subject; and c) implanting an encapsulation device into said implantation site of said subject. Further disclosed herein are methods comprising: a) implanting a pre-vascularization device into an implantation site of a subject; and b) implanting an encapsulation device into said implantation site of said subject. Further disclosed herein are methods comprising: a) implanting an encapsulation device into an implantation site of a subject for a defined period of time, wherein said encapsulation device does not comprise cells; and b) loading said encapsulation device with a plurality of cells after said defined period of time. Further disclosed herein are methods comprising: a) introducing an oxygenation device to an implantation site of a subject for a defined period of time, wherein said oxygenation device is configured to deliver oxygen to said implantation site; and b) implanting an encapsulation device into said implantation site, wherein said encapsulation device comprises a plurality of cells contained within a lumen of said encapsulation device. Further disclosed herein are methods comprising: (i) introducing one or more chemical supplements to an implantation site of a subject and (ii) introducing an encapsulation device to said implantation site, wherein said one or more chemical supplements provide controlled release of oxygen to said implantation site. Further disclosed herein are methods comprising: introducing an encapsulation device to an implantation site of a subject, wherein said encapsulation device comprises: (i) a plurality of therapeutic cells contained within a lumen of said encapsulation device; and (ii) a plurality of supporting cells contained with said lumen of said encapsulation device, wherein said plurality of supporting cells promote oxygenation of said implantation site.

Disclosed herein are devices comprising: an exterior surface comprising polycaprolactone (PCL) polymer, wherein said exterior surface is configured to be in contact with a bodily fluid or a bodily tissue of a subject, and wherein, when said device is implanted in a subject for at least 5 months, a level of fibrosis around said device has a fibrosis score of Grade 2 or less, as measured by histology, and wherein said PCL polymer has a number average molecular weight of 50-200 kDa.

### Definitions

As used herein and in the appended claims, the singular forms “a.” “an.” and “the” include plural referents unless the context clearly dictates otherwise. It is further noted that the claims can be drafted to exclude any optional element. As such, this statement is intended to serve as antecedent basis for use of such exclusive terminology as “solely.” “only.” and the like in connection with the recitation of claim elements, or use of a “negative” limitation.

Certain ranges or numbers are presented herein with numerical values being preceded by the term “about.” The term “about” is used herein to mean plus or minus 1%, 2%, 3%, 4%, or 5% of the number that the term refers to.

As used herein, the terms “subject” and “host” may be used interchangeably and may be any animal, including mammals (e.g., a human or non-human animal).

As used herein, the terms “treat”. “treating”, or “treatment”, include alleviating, abating, or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “connectivity diameter” refers to the diameter of the open space or the channel connecting two adjacent pores. As used herein, the term “pore diameter” refers to the distance between two opposite walls of a pore.

As used herein, the term “encapsulated,” refers to cells that are contained or enclosed within a device of the disclosure. For example, encapsulated cells may refer to cells that are contained within a lumen or an internal space of a device of the disclosure. In another example, encapsulated cells may refer to cells that are contained within a membrane of the device (e.g., embedded within or attached to a macroporous scaffold of the device).

As used herein, the term “lumen” refers to an internal space or volume of a device of a disclosure. In some cases, a lumen is created by sealing the periphery of a first polymer layer and a second polymer layer, thereby generating an internal space or volume for encapsulating cells. The term “lumen” as used herein also encompasses the space where cells embed or attach within the device, for example, the space (e.g., pores, channels, etc.) within a macroporous scaffold of the device.

Headers as used herein in this application are for organizational purposes only. The headers are not intended to limit the disclosed inventive concepts in any way.

Unless defined otherwise, all technical and scientific terms used herein have the same meaning as commonly understood by one of ordinary skill in the art to which the disclosure, e.g., methods and compositions described herein, belong. Although any methods and materials similar or equivalent to those described herein can also be used in the practice or testing of the methods and compositions described herein, representative illustrative methods and materials are now described.

### Encapsulation Devices

Encapsulation devices may be used in conjunction with the oxygenation devices and methods provided herein. Examples of encapsulation devices that may be used herein are described in WO2016/154333, which is herein incorporated by reference in its entirety; however, the oxygenation devices and methods provided herein may be used with any number of encapsulation devices. The encapsulation devices may comprise various designs. In some embodiments, the encapsulation devices may be designed to contain therapeutic cells. In some embodiments, the encapsulation devices may be designed to contain therapeutic cells within a lumen or internal compartment of the encapsulation devices. In some embodiments, the encapsulation devices may be designed such that, when the encapsulation device is transplanted into a biological subject (e.g., a human), the cells contained within the encapsulation device remain viable and/or functional for a period of time. The encapsulation devices may comprise a biocompatible material. In some embodiments, the biocompatible material is a biocompatible polymer. Non-limiting examples of biocompatible polymers include: polycaprolactone, methacrylate polymer, polyethyleneimine, polyethyleneimine-dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapatite, poly(lactic acid), polyhydroxyvalerte and copolymers thereof, polyhydroxy butyrate and copolymers thereof, polycaprolactone, polydiaxanone, polyanhydride, polycyanocrylate, poly(amino acids), poly(orthoesters), polyesters, collagen, gelatin, cellulose polymer, chitosans, alginates, or any combination thereof.

In some embodiments, a device for transplantation of an encapsulated population of cells (e.g., an encapsulation device) into the body of a subject may include a first nanoporous or microporous polymer layer, a second nanoporous or microporous polymer layer, wherein the first and second layers are in contact with each other along a periphery of the first and second layers thereby defining an enclosed space or a lumen between the first and second layers; and a population of cells disposed in the lumen between the first and second layers.

The device may be configured to induce minimal foreign body response and to promote vascularization into the device. The porous material of the thin film device (e.g., the encapsulation device) may allow for exchange of molecules (e.g., diffusion of oxygen, nutrients, cell metabolism waste products, therapeutic proteins, or combinations thereof) via the pores but may be substantially impermeable to cells and to large proteins such as immunoglobulins and may limit the transport of smaller proteins, such as, for example, cytokines, through the pores. The devices disclosed herein may isolate the cells present in the lumen of the device from the immune system of the subject and may allow for exchange of smaller molecules supporting the viability and function of the transplanted cells.

The devices disclosed herein may support viability of cells present in the lumen of the device (e.g., the encapsulation device) upon transplantation into a subject for at least one month, at least 2 months, at least 3 months, at least 4 months, at least 5 months, at least 6 months, at least 7 months, at least 8 months, at least 9 months, at least 10 months, at least 11 months, at least 12 months, at least 2 years, at least 3 years, at least 4 years, at least 5 years, or longer than 5 years.

The device (e.g., the encapsulation device) may be impermeable to cells such that cells may not cross the first and second polymer layers in detectable numbers. The device may be impermeable to immunoglobulins such that the concentration of any immunoglobulins that gain access to the lumen of the device may be below the level needed for an immune response against the cells present inside the lumen of the device. The device may substantially limit cytokines from entering the lumen of the device such that the concentration of cytokines that gain access to the lumen of the device may be below the level required for an immune response against the cells present inside the lumen of the device.

The devices disclosed herein may be planar and may have any two-dimensional planar shape. In certain embodiments, the device may be circular, elliptical, square, rectangular, or a combination thereof. In some embodiments, the device may be substantially circular and may have a diameter between 1 cm and 5 cm. In some embodiments, the device may be substantially circular and may have a diameter between 0.5 cm and 4.5 cm. 2 cm and 6 cm. 3 cm and 7 cm. 4 cm and 8 cm, or 5 cm and 10 cm.

In certain embodiments, the device (e.g., the encapsulation device) may comprise an additional layer for increasing the rigidity of the device. For example, the device may include a layer of material disposed along a side edge of the device. The additional layer may facilitate maintenance of the device as a substantially planar device by preventing folding of the device during placement in a subject and/or after placement in a subject. The layer of material may be the same material as used for the first layer or the second layer or may be of a different material.

In certain embodiments, the device (e.g., the encapsulation device) may be sealed entirely along the edges of the device, thereby forming a completely enclosed internal space or lumen. In other embodiments, the device may be open at one or more locations at an edge of the device, allowing access to the internal space or lumen of the device. When the device comprises two openings into the lumen of the device, the two openings may be located opposite each other, such as, on opposite sides of the planar device.

In certain embodiments, the device may comprise a lumen having one or more openings at which the first and second polymer layers are not sealed together. The device may further comprise a tubing inserted into the opening. In certain embodiments, the tubing may be affixed to the device by sealing the first and second layers, at the opening, to the exterior wall of the tubing. In certain embodiments, a first end of the tubing may be placed at the opening and positioned in the lumen such that a minimal volume of the lumen is taken up by the tubing while allowing loading of fluids into the lumen of the device. The second end of the tubing, which is distal to the first end may be used as a port for introducing fluids into the lumen via the tubing.

In certain embodiments, the device may include two openings at which the first and second layers are not sealed together, thereby defining a lumen with two openings. Each of the openings may comprise a tubing placed at the openings. The first opening may be used as an ingress port for introducing a fluid (e.g., a cell medium containing cells) into the lumen of the device and the second opening may be used as an egress port for removing fluids (e.g., fluid containing dead cells, cell debris, etc.) from the lumen of the device. The length of the tubing placed within the lumen may be held at a minimal to maximize the volume available for the population of cells in the lumen. The tubing may be affixed to the device by sealing the first and second layers of the device to the exterior wall of the tubing at the first ends of each of the tubings. The second end of the tubing forming one or more access ports into the lumen of the device may be closed when not being used to access the interior of the lumen. The second end(s) may be closed by forming a plug at the second end(s). In certain cases, the plug may be a silicone plug.

The length of the tubing may be selected based on a number of factors. For example, a shorter tubing may be used when loading a population of cells into the lumen of the device ex vivo, for example, prior to placement of the device into a subject. A longer tubing may be used if the tubing is to be used for introducing fluids into and/or removing fluids from the lumen of the device after placement of the device into a subject.

In certain cases, the device (e.g., the encapsulation device) may comprise first and second polymer layers that are sealed to each other along the edges other than at two locations creating a lumen defined by the sealed edges and having two openings. A first end of a first tubing may be placed at a first of the two openings. A first end of a second tubing may be placed at a second of the two openings. The first ends of the first and second tubing may be affixed to the device by sealing the first layer and the second layer to the exterior wall of the device, such that the lumen is sealed around the exterior wall of the tubing(s). The second ends of the first and second tubing may be connected to a remote fill port which converts the device into a refillable, closed system. The remote fill port may contain a solid detectable backing, which allows for, e.g., percutaneous locating of the port with a magnet finder if the backing is magnetic. The remote fill port may include two separate chambers in the port for access to the second end of the first tubing and the second end of the second tubing. In certain cases, the tubing may be, e.g., silastic R silicone tubing. The port may also comprise suture tabs at its base to allow for suturing to soft tissue upon implantation, thus helping resist turning or movement of the port. An exemplary remote fill port is described in WO 2016/028774, which is herein incorporated by reference in its entirety.

In certain embodiments, the device and/or the population of cells in the device may be amenable to visualization after placement of the device into the subject. For example, the device and/or cells may be fluorescent and as such may be visualized by imaging a portion of the body of the subject into which the device is placed. A fluorescent device may facilitate removal of the device and/or access to the tubing of the device if the device is placed wholly inside a subject.

The device (e.g., the encapsulation device) may comprise a first layer that may be nanoporous or microporous and a second layer that may be nanoporous or microporous. The microporous and/or the nanoporous layer may be formed as described in U.S. Patent Application Publication No. 20140170204, which is herein incorporated by reference in its entirety.

In certain embodiments, the first and/or the second layer of the device may be microporous. The pore diameter in the microporous layer may range from 1 μm to 5 μm. The pore diameter in the microporous layer may range from 1 μm to 4.5 μm, 1 μm to 3.5 μm, 1 μm to 2.5 μm, 1 μm to 2 μm, 1.5 μm to 3 μm, 1.5 μm to 2.5 μm, 1.5 μm to 3 μm, 1.5 μm to 5 μm. The pore diameter in the microporous layer may be about 1 μm, about 2 μm, or about 3 μm. The pores in the microporous layer may be created by forming a layer that includes a polymer that is water insoluble and a pore forming agent that is a water-soluble polymer. The pore forming agent may form spheres in the water insoluble polymer layer, which spheres may be dispersed along the width of the water insoluble polymer layer as well as across the thickness of the water insoluble polymer layer. The spheres may determine the pore diameter and a plurality of spheres, when connected across the thickness of the water insoluble polymer layer (cross-section of the water insoluble polymer layer), may provide a continuous channel across the water insoluble polymer layer upon dissolution of the spheres. For the formation of a microporous layer, the spheres of the water soluble polymer may be dissolved by exposing the layer to an aqueous solution, thereby creating spaces/holes throughout the microporous membrane. The spaces/holes may have a diameter of about 1 μm to 5 μm. The diameter of the spaces/holes may range from 1 μm to 4.5 μm, 1 μm to 3.5 μm, 1 μm to 2.5 μm, 1 μm to 2 μm, 1.5 μm to 3 μm, 1.5 μm to 2.5 μm, 1.5 μm to 3 μm, 1.5 μm to 5 μm. The diameter of the spaces/holes may be about 1 μm, about 2 μm, or about 3 μm. A series of spaces/holes may be present sequentially across the thickness of the water insoluble polymer layer and interconnected to provide a channel, which channel may have a varying diameter. For example, a region in the channel at which two spaces are connected (e.g., where two spheres of the pore forming agent were in contact with each other and dissolved to provide two spaces connected to each other) may have a smaller channel diameter than the other regions in the channel. The smallest diameter of the channel may be referred to as the connectivity diameter. In certain embodiments, the connectivity diameter may range from 900 nm to 100 nm, such as, 900 nm to 150 nm, 900 nm to 200 nm, 700 nm to 100 nm, 700 nm to 150 nm, 700 nm to 200 nm, 500 nm to 100 nm, 500 nm to 150 nm, 300 nm to 100 nm, 300 nm to 150 nm, or 300 nm to 200 nm.

In certain embodiments, the first and/or the second layer of the device (e.g., the encapsulation device) may be nanoporous. The pore diameter in the nanoporous layer may range from 10 to 300 nm, such as, 20 to 300 nm, 30 to 300 nm, 10 to 200 nm, 20 to 200 nm, 30 to 200 nm, 10 to 100 nm, 20 to 100 nm, 30 to 100 nm, 30 to 50 nm, 30 to 40 nm, 20 to 250 nm, 20 to 150 nm, or 25 to 150 nm, e.g., 30 nm, 50 nm, 100 nm, 150 nm, or 300 nm. In certain embodiments, the nanopore layer may comprise a backing layer that may structurally support the nanopore layer. In certain embodiments, the backing layer may be a microporous layer, such as a microporous layer described herein.

In certain embodiments, the first and/or the second layer of the device may have a percentage (%) porosity of at least 50%, such as, at least 55%, at least 60%, at least 65%, or at least 70%, e.g., from 50% to 70%, 55% to 70%, or 55% to 65%. The percentage of porosity of the first and second nanoporous polymer layers of the device (e.g., the encapsulation device) may be controlled by the number of nanorods used for the fabrication of the layer. Similarly, the percentage of porosity of the first and second microporous polymer layers of the device may be controlled by the amount of pore forming agent (e.g., a water-soluble polymer) used during manufacture of the microporous polymer layer.

As noted herein, the device may be a thin layer device (e.g., the encapsulation device) where the individual first and second layers of the device may be each less than 30 μm in thickness. As such, the first and second layers may be less than 30 μm thick, such as, less than 25 μm, less than 20 μm, less than 15 μm, less than 10 μm, or less than 5 μm, e.g., 20 μm to 10 μm, 25 μm to 20 μm, 15 μm to 10 μm, 13 μm to 10 μm, 13 μm to 8 μm, 12 μm to 8 μm, 11 μm to 9 μm, or 10 μm±5% in thickness.

In certain embodiments, the device (e.g., the encapsulation device) is less than 60 μm thick and has a surface area of 0.5 cm² to 5 cm², such as, a surface area larger than 0.75 cm², larger than 1 cm², larger than 1.5 cm², larger than 2 cm², larger than 2.5 cm², larger than 3 cm², larger than 3.5 cm², larger than 4 cm², larger than 4.5 cm², and up to 5 cm². The device may include a first layer and a second layer. Both layers may be microporous or nanoporous. The diameter of the pores in the microporous polymer layers may range from 1 μm to 5 μm with a connectivity diameter in the range as noted herein. The diameter of the pores in the nanoporous polymer layer may range from 10 nm to 300 nm, such as, 20 nm to 300 nm, 30 to 300 nm, 10 nm to 200 nm, 10 nm to 50 nm, 20 nm to 200 nm, 30 nm to 200 nm, 10 nm to 100 nm, 20 nm to 100 nm, 30 nm to 100 nm, 20 nm to 250 nm, 20 nm to 150 nm, or 25 nm to 150 nm, e.g., 20 nm, 30 nm, 50 nm, 100 nm, 150 nm, or 300 nm.

In certain embodiments, the device (e.g., the encapsulation device) is less than 50 μm thick and has a surface area of 0.75 cm² to 4.5 cm² per side. The diameter of the pores in the microporous polymer layers may range from 1 μm to 3 μm, where the connectivity diameter of the pores (diameter of a through channel) may range from 900 nm to 100 nm, such as, 900 nm to 150 nm, 900 nm to 200 nm, 700 nm to 100 nm, 700 nm to 150 nm, 700 nm to 200 nm, 500 nm to 100 nm, 500 nm to 150 nm, 300 nm to 100 nm, 300 nm to 150 nm, or 300 nm to 200 nm. The diameter of the pores in the nanoporous polymer layers may range from 20 nm to 100 nm, such as, 40 nm to 100 nm, 30 nm to 50 nm, 20 nm to 50 nm, or 25 nm to 100 nm, e.g., 20 nm, 30 nm, 50 nm, or 100 nm.

In certain embodiments, the device (e.g., the encapsulation device) is less than 40 μm thick and has a surface area of 1 cm² to 4 cm² per side. The size of the pores in the microporous polymer layer may range from 1 μm to 3 μm with a connectivity diameter in the range as noted herein. The size of the pores in the nanoporous polymer layers may range from 25 nm to 100 nm, such as, 25 nm to 75 nm, 30 nm to 50 nm, 25 nm to 50 nm, or 25 nm to 40 nm, e.g., 25 nm, 30 nm, 50 nm, or 100 nm.

In certain embodiments, the device (e.g., the encapsulation device) is less than 30 μm thick and has a surface area of 1 cm² to 2 cm² per side. The diameter of the pores in the microporous polymer layer may range from 1 μm to 3 μm with a connectivity diameter in the range as noted herein. The diameter of the pores in the nanoporous polymer layers may range from 30 nm to 100 nm, such as, 30 nm to 75 nm, 30 nm to 50 nm, 40 nm to 100 nm, 40 nm to 75 nm, 40 nm to 50 nm, 50 nm to 100 nm, or 50 nm to 75 nm, e.g., 30 nm, 40 nm, 50 nm, 60 nm, or 80 nm. For example, the first and second layers may each be 10 μm±3 μm thick. In certain embodiments, the first layer may be microporous and the second layer may be nanoporous. In certain embodiments, the first layer may be nanoporous and the second layer may be microporous.

In certain embodiments, the device (e.g., the encapsulation device) comprises a first layer and a second layer, each of which are each 10 μm±3 μm thick, wherein the device has a surface area of 1 cm² to 2 cm² per side. The first layer and the second layer may be nanoporous and the diameter of the pores in the nanoporous polymer layers may range from 30 nm to 100 nm, such as, 30 nm to 75 nm, 30 nm to 50 nm, 40 nm to 100 nm, 40 nm to 75 nm, 40 nm to 50 nm, 50 nm to 100 nm, or 50 nm to 75 nm, e.g., 30 nm, 40 nm, 50 nm, 60 nm, or 80 nm. In other embodiments, the first layer and the second layer may be microporous and the diameter of the pores in the microporous polymer layer may range from 1 μm to 3 μm (e.g., 2 μm±0.5 μm diameter) with a connectivity diameter ranging from 900 nm to 100 nm, such as, 900 nm to 150 nm, 900 nm to 200 nm, 700 nm to 100 nm, 700 nm to 150 nm, 700 nm to 200 nm. 500 nm to 100 nm. 500 nm to 150 nm. 300 nm to 100 nm. 300 nm to 150 nm, or 300 nm to 200 nm.

The devices of the present disclosure may be configured to prevent immune cells from entering the lumen of the device (e.g., the encapsulation device) and substantially inhibit antibodies and cytokines from entering the lumen while promoting nutrient exchange with the encapsulated cells and release of therapeutic proteins secreted by the encapsulated cells as well as diffusion of metabolic waste products out of the device. The pores and the thickness of the thin layers forming the thin film device (e.g., the encapsulation device) of the present disclosure may be configured to permit passage of small molecules, such as salts, sugars, amino acids, dopamine, glucose, insulin and substantially inhibit passage of large molecules such as, antibodies. C3b, cytokines (e.g., interferons, interleukins, tumor necrosis factors, and the like) and of cells. The first and second porous polymer layers may be configured to allow exchange of small molecules that have a molecular weight less than 10 kDa and/or a hydrodynamic radius of less than 2 nm. The first and second porous polymer layers may be configured to substantially limit large molecules that have a molecular weight greater than 15 kDa and/or a hydrodynamic radius of greater than 2 nm from crossing the layers and entering the lumen of the device.

Any polymer material may be used to form the first and second porous layers of the devices (e.g., the encapsulation device) disclosed herein. Representative polymers include, but are not limited to, methacrylate polymers, polyethylene-imine and dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapatite, poly(lactic acid), polyhydroxyvalerte and copolymers, polyhydroxy butyrate and copolymers, polycaprolactone, polydiaxanone, polyanhydrides, polycyanocrylates, poly(amino acids), poly(orthoesters), polyesters, collagen, gelatin, cellulose polymers, chitosans, and alginates or combinations thereof. Additional examples that may be used to coat the scaffold include, but are not limited to, collagen, fibronectin, extracellular matrix proteins, vinculin, agar, and agarose. Various mixture of the polymers may be used.

In certain embodiments, the first layer and/or the second layer may be formed from poly(caprolactone) (PCL). The PCL used for forming the first and/or the second layer may have a number average molecular weight (Mn) higher than 50 kDa, such as, higher than 55 kDa, 60 kDa, 65 kDa, or 70 kDa, and up to 200 kDa, e.g., 50 kDa to 200 kDa, 55 kDa to 200 kDa, 60 kDa to 200 kDa, 65 kDa to 200 kDa, 70 kDa to 200 kDa, 50 kDa to 150 kDa, 55 kDa to 150 kDa, 60 kDa to 150 kDa, 65 kDa to 150 kDa, 70 kDa to 150 kDa, 50 kDa to 100 kDa, 55 kDa to 100 kDa, 60 kDa to 100 kDa, 65 kDa to 100 kDa, 70 kDa to 100 kDa, 50 kDa to 90 kDa. 55 kDa to 90 kDa, 60 kDa to 90 kDa, 65 kDa to 90 kDa, or 70 kDa to 90 kDa. Furthermore, the molecular weight of the PCL polymer may be selected based on the duration for which the device (e.g., the encapsulation device) is to be maintained in the body of a subject (e.g., a human) without substantial degradation of the polymer.

Under physiological conditions, a biodegradable polymer such as PCL polymer may degrade by random chain scission, which may give rise to a two-phase degradation. Initially, as molecular weight decreases the physical structure may not be significantly affected. Degradation may take place throughout the polymer material, and may proceeds until a critical molecular weight is reached, when degradation products become small enough to be solubilized. At this point, the structure may start to become significantly more porous and hydrated. In certain cases, a higher molecular weight polymer may be used when the device (e.g., the encapsulation device) is to be used for providing transplanted cells into the subject for at least one year. For example, when the device is to maintain the integrity of the porous polymer layers for at least one year, the porous polymer layers may be made from a polymer, that has at least 70 kDa Mn, e.g., at least 75 kDa Mn, at least 80 kDa Mn, at least 85 kDa Mn. or at least 90 kDa Mn, and up to 100 kDa Mn. In embodiments where the device may be configured to degrade by 6 months, the first and second porous polymer layers may be formed from a lower molecular weight polymer, such as, a polymer having Mn of 10 kDa to 20 kDa.

In some embodiments, the biodegradable polymer includes a blend of polymers where the polymers may be of the same or a different type of polymer, and each polymer may be of a different molecular weight (MW). In some embodiments, the biodegradable polymer may include a blend of a high MW polymer and a low MW polymer. The high MW polymer may be of about 25 kDa or more, such as about 30 kDa or more, about 40 kDa or more, about 50 kDa or more, about 60 kDa or more, about 70 kDa or more, about 80 kDa or more, about 90) kDa or more, or about 100 kDa, and up to 150 kDa. The low MW polymer may be of about 20 kDa or less, such as about 15 kDa or less, about 10 kDa or less, about 8 kDa or less, about 6 kDa or less, and down to 4 kDa.

In some embodiments, the ratio by mass of the high MW polymer to the low MW polymer in a blend of polymers may be between about 1:9 and about 9:1, such as between about 2:8 and about 8:2, between about 2:8 and about 6:4, or between about 2:8 and about 1:1. In certain embodiments, the ratio by mass of the high MW polymer to the low MW polymer may be about 3:17, about 2:8, about 1:3, about 3:7, about 7:13, about 2:3, about 9:11, about 1:1, about 11:9, or about 3:2.

In certain embodiments, thin film devices for transplantation of cells (e.g., the encapsulation device) into the body of a subject do not comprise poly(lactic-co-glycolic acid) (PLGA), polyvinylidene difluoride (PVDF), alginate, collagen, gelatin, agarose, silicon, cellulose phosphate, or polypropylene (PP).

In certain embodiments, the exterior surface of the device may be modified by disposing one or more agents that improve the device. In other embodiments, the inside surface of the device may be modified by disposing one or more agents that improve the device. In yet other embodiments, the device may be modified within the membrane by disposing one or more agents that improve the device. For example, molecules or factors that promote vascularization of the device or inhibit immune or inflammatory response to the device may be disposed on the exterior of the device, the inside surface of the device, or within the membrane of the device, or any combination thereof. Such molecules include, but are not limited to: VEGF (vascular endothelial growth factor), PDGF (platelet-derived growth factor). FGF-1 (fibroblast growth factor), angiopoietin MCP-1, avf33, avf35, CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like. The molecules may include vascular inducing molecules secreted by the endocrine tissue (e.g., Parathyroid, etc.). The molecules may include engraftment supporting molecules secreted by the endocrine tissue (e.g., Parathyroid, etc.). The molecules may help the encapsulated tissue. The molecules may help the graft site. The molecules may help the encapsulated tissue and the graft site. The molecules may include vascular inducing and/or engraftment supporting molecules secreted by endocrine tissues (e.g., Parathyroid, etc.) that may help the encapsulated tissue and/or the graft site. The molecules or factors may secrete or promote. The molecules or factors may be secreted or promoted. Some examples of molecules or factors include, but are not limited to: (VEGF (vascular endothelial growth factor). PDGF (platelet-derived growth factor). FGF-1 (fibroblast growth factor), angiopoietin MCP-1, avf33, avf35. CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like. Non-limiting examples of mammalian cell types that may be used include, but is not limited to: thyroid cells, parathyroid cells, bone marrow cells, mesenchymal stem cells, stromal cells, pluripotent stem cells, induced pluripotent stem cells, embryonic stem cells, blood vessel cells, cells derived from adipose tissue, cells derived from bone marrow, intestinal cells or cells derived therefrom, islets or islet cells. Sertoli cells, beta cells, progenitors of islet cells, progenitors of beta cells, peripheral blood progenitor cells, stem cells or derivatives of stem cells or stem cells products. Cells may be derived and/or isolated from adult tissue, retinal progenitor derivative cells, cardiac progenitor derivative cells, osteoprogenitor cells, neuronal progenitor cells, genetically transformed cells, or any combination thereof. In some embodiments, factors may be delivered from the cell types described herein. In some embodiments, the factors may promote cellular activity. In some embodiments, the factors may promote cellular response. The factors may include, but are not limited to: VEGF (vascular endothelial growth factor). PDGF (platelet-derived growth factor). FGF-1 (fibroblast growth factor), angiopoietin MCP-1, avf33, avf35. CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like. The factors may include vascular inducing factors secreted by the endocrine tissue (e.g., Parathyroid, etc.). The factors may include engraftment supporting factors secreted by the endocrine tissue (e.g., Parathyroid, etc.). The factors may help the encapsulated tissue. The factors may help the graft site. The factors may help the encapsulated tissue and the graft site. The factors may include vascular inducing and/or engraftment supporting factors secreted by endocrine tissues (e.g., Parathyroid, etc.) that may help the encapsulated tissue and/or the graft site. The factors may help secrete or promote. Some examples of factors that may help secrete or promote include, but are not limited to: VEGF (vascular endothelial growth factor). PDGF (platelet-derived growth factor). FGF-1 (fibroblast growth factor), angiopoietin MCP-1, avf33, avf35. CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like.

The devices (e.g., the encapsulation device) of the present disclosure may be used to provide encapsulated cells in a subject. The method for providing the encapsulated cells includes providing a thin film device, e.g., a multilayer thin film device, as provided herein, transplanting the thin film device into the subject, promoting vascularization into the lumen of the device via the pores in the first polymer layer and/or second polymer layer: limiting foreign body response to the device: limiting ingress of cells, immunoglobulins, and cytokines into the lumen via the first and the second polymer layers; and releasing from the first polymer layer and/or the second polymer layer molecules secreted by the population of cells. The devices (e.g., the encapsulation device) disclosed herein may promote vascularization into the lumen of the device such that at least 20% of the device is vascularized within a month after transplantation into a subject, such as at least a 30% vascularization, 40% vascularization, 50% vascularization. 60% vascularization within a month after transplantation into a subject. The devices disclosed herein may limit the diffusion of cytokines and immunoglobulins through the pores in the first polymer layer and second polymer layer such that the diffusion rate is less than 50%, less than 40%, less than 30%, less than 20% compared to diffusion in absence of a barrier layer.

The devices (e.g., the encapsulation device) of the present disclosure may be sized to house an effective number for transplanted cells for treatment of a subject in need thereof. For example, the subject may be suffering from a condition caused by lack of functional cells, e.g., wherein molecules typically secreted by functional cells are not secreted or are secreted at a level resulting in the condition. Providing functional cells may alleviate the condition. Exemplary conditions include type 1 diabetes. Parkinson's disease, muscular dystrophy and the like.

The device (e.g., the encapsulation device) may be transplanted into any suitable location in the body of subject (e.g., a human), such as, subcutaneously, intraperitoneally, or in the brain, spinal cord, pancreas, liver, uterus, skin, bladder, kidney, muscle and the like. The site of implantation may be selected based on the diseased/injured tissue that requires treatment. For treatment of a disease such as diabetes mellitus (DM), the device (e.g., the encapsulation device) may be placed in a clinically convenient site such as the subcutaneous space or the omentum.

Populations of cells for transplantation using the devices described herein include but are not limited to, bone marrow cells: mesenchymal stem cells, stromal cells, pluripotent stem cells (e.g., induced pluripotent stem cells and embryonic stem cells), blood vessel cells, precursor cells derived from adipose tissue, bone marrow derived progenitor cells, intestinal cells, islets. Sertoli cells, beta cells, progenitors of islets, progenitors of beta cells, peripheral blood progenitor cells, stem cells isolated from adult tissue, retinal progenitor cells, cardiac progenitor cells, osteoprogenitor cells, neuronal progenitor cells, and genetically transformed cells, or a combination thereof. The population of cells may be a single population of cells or a mixed population of cells. For example, the population of cells may be one or more of the population of cells described herein. In some embodiments, the mixed population of cells may increase transplanted cell survival. In some embodiments, the mixed population of cells may help improve cell engraftment. In some embodiments, the mixed population of cells may help increase transplanted cell survival or improve cell engraftment, or combinations thereof. In some embodiments, the mixed population of cells may result in secreted factors. In some embodiments, factors may be delivered from the cell types described herein. In some embodiments, the factors may promote cellular activity. In some embodiments, the factors may promote cellular response. The factors may include, but are not limited to: VEGF (vascular endothelial growth factor). PDGF (platelet-derived growth factor). FGF-1 (fibroblast growth factor), angiopoietin MCP-1, avf33, avf35. CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like. The factors may include vascular inducing factors secreted by the endocrine tissue (e.g., Parathyroid, etc.). The factors may include engraftment supporting factors secreted by the endocrine tissue (e.g., Parathyroid, etc.). The factors may help the encapsulated tissue. The factors may help the graft site. The factors may help the encapsulated tissue and the graft site. The factors may include vascular inducing and/or engraftment supporting factors secreted by endocrine tissues (e.g., Parathyroid, etc.) that may help the encapsulated tissue and/or the graft site. The factors may help secrete or promote. Some examples of factors that may help secrete or promote include, but are not limited to: VEGF (vascular endothelial growth factor). PDGF (platelet-derived growth factor). FGF-1 (fibroblast growth factor), angiopoietin MCP-1, avf33, avf35. CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like.

The secreted factors may come from one or more than one of the population of cells within the mixed population of cells. The secreted factors may come from the mixture in the mixed population of cells. The secreted factors may help increase cell survival and improve cell engraftment. The encapsulated cells may be from the subject (e.g., autologous cells), from another donor (e.g., allogeneic cells) or from other species (e.g., xenogeneic cells). The cells may be introduced into the lumen of the device and the device may be immediately (e.g., within a day) implanted into a subject or the cells may be cultured for longer periods (e.g., greater than one day) to allow for cell proliferation prior to implantation. The number of cells introduced into the lumen of the device may vary and may be determined empirically.

In certain embodiments, the devices disclosed herein may be used to treat a person having diabetes, such as, type 1 diabetes. The device may include pancreatic islet cells or may include stem cells that are capable of differentiating into pancreatic islet cells. In certain embodiments, pluripotent stem cells (PSCs) may be differentiated into pancreatic islet cells inside the device and then the device containing the differentiated pancreatic islet cells may be placed in the subject. In some embodiments, the device may be placed in the omentum. In some embodiments, the device may be placed adjacent to the pancreas or liver. In some cases, the devices may be implanted into a subject by subcutaneous implantation, intramuscular implantation, subdermal implantation, intraperitoneal implantation, retroperitoneal implantation, omentum implantation, implantation into the brain, subfacial implantation, implantation into the bone, ocular implantation, implantation into an organ, implantation adjacent to an organ (e.g., liver, etc.). In some case, the device may include PSCs and the device may be implanted adjacent the pancreas or liver of the subject.

As noted herein, the devices disclosed herein may maintain the transplanted cells in a functional and viable state for at least 1 month and up to a period of at least 2 months, 3 months. 4 months. 5 months. 6 months. 7 months. 8 months. 9 months. 10 months, 11 months, or up to a year or longer. In certain cases, the integrity of the device may be maintained for at least one year and the cells in the device may be replaced with a fresh population of cells using the tubing attached to the device (e.g., via a remote fill port) while the device is located inside a subject.

The methods and devices disclosed herein may be used for both human clinical and veterinary applications. Thus, the subject or patient to whom the device is administered can be a human or, in the case of veterinary applications, can be a laboratory, agricultural, domestic, or wild animal. The subject devices and methods can be applied to animals including, but not limited to, humans, laboratory animals such as monkeys and chimpanzees, domestic animals such as dogs and cats, agricultural animals such as cows, horses, pigs, sheep, goats, and wild animals in captivity such as bears, pandas, lions, tigers, leopards, elephants, zebras, giraffes, gorillas, dolphins, and whales.

In some aspects of the disclosure, the device (e.g., the encapsulation device) may comprise a first polymer layer. In some cases, the first polymer layer may be a polymer membrane or a polymer film. The first polymer layer may comprise a plurality of pores. In some cases, the first polymer layer may be a nanoporous polymer layer.

In some cases, the plurality of pores present in the first polymer layer may have an average connectivity diameter. In some cases, the plurality of pores present in the first polymer layer may have an average connectivity diameter ranging from about 10 nm to about 2 μm. For example, the plurality of pores present in the first polymer layer may have an average connectivity diameter ranging from about 20 nm to about 300 nm, from about 30 nm to about 300 nm, from about 10 nm to about 200 nm, from about 20 nm to about 200 nm, from about 30) nm to about 200 nm, from about 10 nm to about 100 nm, from about 20 nm to about 100 nm, from about 30 nm to about 100 nm, from about 30 nm to about 50 nm, from about 30 nm to about 40) nm, from about 20 nm to about 250) nm, from about 20 nm to about 150 nm, from about 25 to about 150 nm, from about 200 nm to about 500 nm, from about 350 nm to about 500 nm, from about 500 nm to about 1.5 μm, or from about 1.0 μm to about 2.0 μm. In some cases, the plurality of pores in the first polymer layer may have an average connectivity diameter of about 10 nm, about 20 nm, about 30 nm, about 40 nm, about 50) nm, about 60) nm, about 70 nm, about 80 nm, about 100 nm, about 110 nm, about 120 nm, about 130) nm, about 140) nm, about 150) nm, about 160 nm, about 170 nm, about 180 nm, about 190 nm, about 200 nm, about 210) nm, about 220) nm, about 230) nm, about 240) nm, about 250) nm, about 260) nm, about 270) nm, about 280) nm, about 290) nm, about 300) nm, about 310 nm, about 320 nm, about 330) nm, about 340) nm, about 350) nm, about 360) nm, about 370) nm, about 380) nm, about 390) nm, about 400 nm, about 410 nm, about 420) nm, about 430) nm, about 440) nm, about 450) nm, about 460) nm, about 470) nm, about 480) nm, about 490 nm, about 500 nm, about 550 nm, about 600 nm, about 650) nm, about 700 nm, about 750) nm, about 800 nm, about 850 nm, about 900 nm, about 950 nm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, or about 2.0 μm. In one aspect, the average connectivity diameter may be about 200 nm. In another aspect, the average connectivity diameter may be about 2.0 μm.

In various aspects, the first polymer layer may have an average connectivity diameter of less than about 2.0 μm, less than about 1.9 μm, less than about 1.8 μm, less than about 1.7 μm, less than about 1.6 μm, less than about 1.5 μm, less than about 1.4 μm, less than about 1.3 μm, less than about 1.2 μm, less than about 1.1 μm, less than about 1.0 μm, less than about 975 nm, less than about 950 nm, less than about 925 nm, less than about 900 nm, less than about 875 nm, less than about 850 nm, less than about 825 nm, less than about 800 nm, less than about 775 nm, less than about 750) nm, less than about 725 nm, less than about 700 nm, less than about 675 nm, less than about 650 nm, less than about 625 nm, less than about 600 nm, less than about 575 nm, less than about 550) nm, less than about 525 nm, less than about 500 nm, less than about 475 nm, less than about 450) nm, less than about 425 nm, less than about 400) nm, less than about 375 nm, less than about 350) nm, less than about 325 nm, less than about 300) nm, less than about 275 nm, less than about 250) nm, less than about 225 nm, less than about 200 nm, less than about 175 nm, less than about 150 nm, less than about 125 nm, less than about 100 nm, less than about 75 nm, less than about 50 nm, less than about 25 nm, less than about 20 nm, or less than about 10 nm.

In some aspects of the disclosure, the plurality of pores present in the first polymer layer may have an average pore diameter. In some cases, the average pore diameter may range from about 1 μm to about 5 μm. For example, the plurality of pores present in the first polymer layer may have an average pore diameter ranging from about 1 μm to about 4.5 μm, from about 1 μm to about 4 μm, from about 1 μm to about 3.5 μm, from about 1 μm to about 3 μm, from about 1 μm to about 2.5 μm, from about 1 μm to about 2 μm, from about 1.5 μm to about 5 μm, from about 2 μm to about 5 μm, from about 2.5 μm to about 5 μm, from about 3 μm to about 5 μm, from about 3.5 μm to about 5 μm, or from about 4 μm to about 5 μm. In some cases, the plurality of pores present in the first polymer layer may have an average pore diameter of about 1 μm, about 1.5 μm, about 2 μm, about 2.5 μm, about 3 μm, about 3.5 μm, about 4 μm, about 4.5 μm, or about 5 μm. In some cases, the first polymer layer may have an average pore diameter of about 2 μm.

In various aspects of the disclosure, the average pore diameter may be less than about 5 μm, less than about 4.5 μm, less than about 4 μm, less than about 3.5 μm, less than about 3 μm, less than about 2.5 μm, less than about 2 μm, less than about 1.5 μm, or less than about 1 μm.

In some cases, the plurality of pores present in the first layer may have an average connectivity diameter that is smaller than the average pore size. In other cases, the plurality of pores present in the first layer may have an average connectivity diameter that is the same or substantially the same as the average pore size.

The first polymer layer may have a thickness. In some cases, the first polymer layer may have a thickness of about 200 μm or less, e.g., about 200 μm, about 190 μm, about 180 μm, about 170 μm, about 160 μm, about 150 μm, about 140 μm, about 130 μm, about 120 μm, about 110 μm, about 100 μm, about 90 μm, about 80 μm, about 70 μm, about 60 μm, about 50 μm, about 40 μm, about 30 μm, about 29 μm, about 28 μm, about 27 μm, about 26 μm, about 25 μm, about 24 μm, about 23 μm, about 22 μm, about 21 μm, about 20 μm, about 19 μm, about 18 μm, about 17 μm, about 16 μm, about 15 μm, about 14 μm, about 13 μm, about 12 μm, about 11 μm, or about 10 μm. In some cases, the thickness of the first polymer layer may be less than about 10 μm. In some cases, the thickness of the first polymer layer may be greater than about 10 μm but less than about 30 μm. In some cases, the thickness of the first layer may be greater than about 18 μm but less than about 22 μm. In some cases, the thickness of the first polymer layer may be greater than about 30 μm. In some cases, the thickness of the first polymer layer may be from about 10 μm to about 200 μm, from about 20 μm to about 50 μm, from about 50 μm to about 300 μm, or from about 100 μm to about 500 μm. In some cases, the thickness of the first polymer layer may be from about 10 μm to about 50 μm. In some cases, the thickness of the first polymer layer is about 20 μm.

The first polymer layer may comprise a biocompatible polymer. Biocompatible polymers may include, without limitation, polycaprolactone (PCL), polytetrafluoroethylene (PTFE), polyethylene (PE), polyvinylidene fluoride (PVDF), methacrylate polymers, polyethylene-imine and dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapatite, poly(lactic acid), polyhydroxyvalerte and copolymers, polyhydroxy butyrate and copolymers, polydiavanone, polyanhydrides, polycyanocrylates, poly(amino acids), poly(orthoesters), polyesters, silicone, collagen, gelatin, cellulose polymers, chitosans, laminin, and alginates, or combinations thereof. In some cases, the first polymer layer may comprise polycaprolactone. In some cases, the biocompatible polymer may be biodegradable (e.g., dissolvable in a biological environment). In some cases, the first polymer layer may be manufactured by weaving, by casting, by extrusion, by deposition, by an emulsion process, and the like. In other cases, the first polymer layer may be manufactured by electrospinning processes or templating processes.

In some aspects of the disclosure, the device (e.g., the encapsulation device) may comprise a second polymer layer. In some cases, the second polymer layer may be a polymer membrane or a polymer film. The second polymer layer may comprise a plurality of pores. In some cases, the second polymer layer may be a macroporous polymer layer. In some cases, the second polymer layer may be a sponge or sponge-like. In some cases, the second polymer layer may be a mesh.

The plurality of pores present in the second polymer layer may have an average connectivity diameter. In some cases, the plurality of pores present in the second polymer layer may have an average connectivity diameter ranging from about 50 μm to about 3 mm. For example, the plurality of pores present in the second layer may have an average connectivity diameter ranging from about 50 μm to about 1 mm, from about 200 μm to about 1.5 mm, from about 300 μm to about 2 mm, from about 400 μm to about 2.5 mm, from about 500 μm to about 3 mm, from about 100 μm to about 500 μm, from about 500 μm to about 1.5 mm, or from about 1.5 mm to about 3 mm. In some cases, the plurality of pores present in the second polymer layer may have an average connectivity diameter of about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, or about 3.0 mm.

In some cases, the second polymer layer may have an average connectivity diameter of greater than about 50 μm, greater than about 100 μm, greater than about 150 μm, greater than about 200 μm, greater than about 250 μm, greater than about 300 μm, greater than about 350 μm, greater than about 400 μm, greater than about 450 μm, greater than about 500 μm, greater than about 550 μm, greater than about 600 μm, greater than about 650 μm, greater than about 700 μm, greater than about 750 μm, greater than about 800 μm, greater than about 850 μm, greater than about 900 μm, greater than about 950 μm, greater than about 1.0 mm, greater than about 1.1 mm, greater than about 1.2 mm, greater than about 1.3 mm, greater than about 1.4 mm, greater than about 1.5 mm, greater than about 1.6 mm, greater than about 1.7 mm, greater than about 1.8 mm, greater than about 1.9 mm, greater than about 2.0 mm, greater than about 2.1 mm, greater than about 2.2 mm, greater than about 2.3 mm, greater than about 2.4 mm, greater than about 2.5 mm, greater than about 2.6 mm, greater than about 2.7 mm, greater than about 2.8 mm, greater than about 2.9 mm, or greater than about 3.0 mm.

The plurality of pores present in the second polymer layer may have an average pore diameter. In some cases, the plurality of pores present in the second polymer layer may have an average pore diameter ranging from about 100 μm to about 3 mm. For example, the plurality of pores present in the second layer may have an average pore diameter ranging from about 100 μm to about 1 mm, from about 200 μm to about 1.5 mm, from about 300 μm to about 2 mm, from about 400 μm to about 2.5 mm, from about 500 μm to about 3 mm, from about 100 μm to about 500 μm, from about 500 μm to about 1.5 mm, or from about 1.5 mm to about 3 mm. In some cases, the plurality of pores present in the second polymer layer may have an average pore diameter of about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, or about 3.0 mm. The plurality of pores present in the second polymer layer may be organized stochastically.

In some cases, the second polymer layer may have an average pore diameter of greater than about 100 μm, greater than about 150 μm, greater than about 200 μm, greater than about 250 μm, greater than about 300 μm, greater than about 350 μm, greater than about 400 μm, greater than about 450 μm, greater than about 500 μm, greater than about 550 μm, greater than about 600 μm, greater than about 650 μm, greater than about 700 μm, greater than about 750 μm, greater than about 800 μm, greater than about 850 μm, greater than about 900 μm, greater than about 950 μm, greater than about 1.0 mm, greater than about 1.1 mm, greater than about 1.2 mm, greater than about 1.3 mm, greater than about 1.4 mm, greater than about 1.5 mm, greater than about 1.6 mm, greater than about 1.7 mm, greater than about 1.8 mm, greater than about 1.9 mm, greater than about 2.0 mm, greater than about 2.1 mm, greater than about 2.2 mm, greater than about 2.3 mm, greater than about 2.4 mm, greater than about 2.5 mm, greater than about 2.6 mm, greater than about 2.7 mm, greater than about 2.8 mm, greater than about 2.9 mm, or greater than about 3.0 mm.

The second polymer layer may have a thickness. In some cases, the second polymer layer has a thickness from about 200 μm to about 1 mm. For example, the second polymer layer may have a thickness from about 200 μm to about 500 μm, from about 250 μm to about 450 μm, from about 300 μm to about 500 μm, from about 350 μm to about 600 μm, from about 400 μm to about 800 μm, from about 550 μm to about 950 μm, from about 500 μm to about 1 mm. In some cases, the second polymer layer may have a thickness of about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1 mm. In some cases, the second polymer layer may have a thickness of about 500 μm.

In some cases, the second polymer layer may have a thickness from about 1 mm to about 5 mm. For example, the second polymer layer may have a thickness from about 1 mm to about 5 mm, from about 2 mm to about 4 mm, about 3 mm to about 5 mm, from about 1 mm to about 3 mm, from about 2.5 mm to about 5 mm, or from about 1.5 mm to about 4.5 mm. In some cases the second polymer layer may have a thickness of about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0) mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4, mm, about 4.5 mm, about 4.6 mm, about 4.7 mm, about 4.8 mm, about 4.9 mm, or about 5.0 mm. In some cases, the second polymer layer may have a thickness of about 2 mm.

The second polymer layer may have a pore density. In some cases, the second polymer layer may have a pore density from about 0.01 g/cm³ to about 0.1 g/cm³. For example, the second polymer layer may have a pore density of about 0.01 g/cm³, about 0.02 g/cm³, about 0.03 g/cm³, about 0.04 g/cm³, about 0.05 g/cm³, about 0.06 g/cm³, about 0.07 g/cm³, about 0.08 g/cm³, about 0.09 g/cm³, or about 0.1 g/cm³.

The second polymer layer may comprise a biocompatible polymer. Biocompatible polymers may include, without limitation, polycaprolactone (PCL), polytetrafluoroethylene (PTFE), polyethylene (PE), polyvinylidene fluoride (PVDF), methacrylate polymers, polyethylene-imine and dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapatite, poly(lactic acid), polyhydroxyvalerte and copolymers,

polyhydroxy butyrate and copolymers, polydiaxanone, polyanhydrides, polycyanocrylates, poly(amino acids), poly(orthoesters), polyesters, collagen, gelatin, cellulose polymers, chitosans, laminin, and alginates, or combinations thereof. In some cases, the second polymer layer may comprise polycaprolactone (PCL). In some cases, the biocompatible polymer may be biodegradable (e.g., dissolvable in a biological environment). In some cases, the second polymer layer may be manufactured by weaving, by extrusion, by casting, by deposition, by an emulsion process, and the like. In other cases, the second polymer layer may be manufactured by electrospinning processes or templating processes.

In some aspects, the second polymer layer may act as a scaffold for biological cells within the lumen of the device (e.g., the encapsulation device). For example, biological cells contained within the lumen of the device may attach to the surface of and/or embed within the pores or channels of the second polymer layer. In some cases, the surface of the second polymer layer may be coated with one or more agents (e.g., a growth factor) that promote survival and/or growth and/or protection of the biological cells within the device. In some cases, the second polymer layer may promote vascularization of the cells encapsulated within the device (e.g., the encapsulation device). For example, the second polymer layer may allow the blood vessels to grow into or penetrate the second polymer layer, such that the blood vessels are in close proximity to the biological cells encapsulated within the device. The blood vessels penetrating the second polymer layer may provide a supply of oxygen and/or nutrients to the cells encapsulated within the device and may promote viability of the cells within the device.

In some cases, the devices (e.g., the encapsulation device) of the disclosure may increase the viability of encapsulated cells relative to transplanted naked cells (e.g., cells not encapsulated within a device). The devices described herein may be particularly well suited for encapsulating cells that have a preference for attaching to or embedding within a substrate, for example, cells that have a preferred three-dimensional architecture or arrangement. In particular examples, the macroporous scaffold of the device may provide a substrate for cells to embed or attach thereon. In some cases, the encapsulated cells embedded within or attached to the macroporous scaffold may have increased viability as compared to encapsulated cells that are not embedded or attached to a scaffold (e.g., free-floating in a lumen of a device).

In various aspects, the first polymer layer and the second polymer layer may each be non-laminated polymer layers. In other words, each of the first polymer layer and the second polymer layer may comprise of a single polymer layer.

In some aspects, the device (e.g., the encapsulation device) of the disclosure may further comprise a third polymer layer. In some cases, the third polymer layer may comprise a plurality of pores. In some cases, the plurality of pores may have average connectivity diameters and/or average pore diameters of those disclosed for the first polymer layer. In some cases, the third polymer layer may be a nanoporous polymer layer. In some cases, the first and third polymer layers may have the same or similar average connectivity diameters and/or average pore diameters. In some cases, the first and third polymer layers may have different average connectivity diameters and/or average pore diameters. In some cases, the third polymer layer may be composed of a biocompatible polymer, such as any biocompatible polymer disclosed for the first polymer layer. In some cases, the first and third polymer layers are composed of the same biocompatible polymer material. In some cases, the first and third polymer layers are composed of different biocompatible polymer materials.

In such devices (e.g., the encapsulation device) comprising a third polymer layer, the third polymer layer may be in contact at a periphery of the device with the first polymer layer, or the third polymer layer may be in contact at the periphery of the device with the first polymer layer and the second polymer layer, thereby forming an enclosed space or lumen between the first polymer layer and the third polymer layer. In such cases, the second polymer layer may be enclosed within the device (e.g., within the lumen, or lining the third polymer layer).

In various aspects, a device (e.g., the encapsulation device) of the disclosure may have a total thickness of about 200 μm to about 5 mm. For example, a device of the disclosure may have a total thickness of about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, about 4.6 mm, about 4.7 mm, about 4.8 mm, about 4.9 mm, or about 5.0 mm.

In some cases, a device (e.g., the encapsulation device) of the disclosure may have a surface area of about 1 cm² to about 100 cm². For example, a device of the disclosure may have a surface area of about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70) cm², about 75 cm², about 80 cm², about 85 cm², about 90 cm², about 95 cm², or about 100 cm².

In some cases, the devices (e.g., the encapsulation device) provided herein may have a two-dimensional planar shape. For example, the two-dimensional planar shape may be, without limitation, circular, elliptical, square, or rectangular. In some cases, the devices (e.g., the encapsulation device) provided herein may have a three-dimensional shape. For example, the three-dimensional shape may be, without limitation, spherical, conical, cuboidal, cylindrical, triangular, hexagonal, tetrahedrical, pyramidal, or octagonal.

In some cases, the devices (e.g., the encapsulation device) provided herein may be used to transplant a variety of different cell types into a biological subject. In some cases, the biological cells may be eukaryotic cells. In some cases, the biological cells may be prokaryotic cells. The biological cells may be derived from any source. In some cases, the cells may be bacterial cells, yeast cells, insect cells, or fungal cells. In some cases, the cells may be mammalian cells. Non-limiting examples of mammalian cell types that may be used include, but is not limited to: thyroid cells, parathyroid cells, bone marrow cells, mesenchymal stem cells, stromal cells, pluripotent stem cells, induced pluripotent stem cells, embryonic stem cells, blood vessel cells, cells derived from adipose tissue, cells derived from bone marrow, intestinal cells or cells derived therefrom, islets or islet cells. Sertoli cells, beta cells, progenitors of islet cells, progenitors of beta cells, peripheral blood progenitor cells, stem cells or derivatives thereof isolated from adult tissue, retinal progenitor derivative cells, cardiac progenitor derivative cells, osteoprogenitor cells, neuronal progenitor cells, genetically transformed cells, or any combination thereof. In some cases, the cells may be islets or islet cells. In some cases, the cells may be beta-cells or beta-like cells. In some cases, the cells may be stem cells. Derivative cells may include isolated subsets of primary tissue cells in an unaltered state or modified through culture conditions or introduction of materials intracellularly. In some cases, the cells may be allogeneic cells. In some cases, the cells may be autologous cells. In some cases, the cells may be xenogeneic cells. In some cases, the cells may be cells that have been engineered to express one or more recombinant proteins.

In some cases, the cells may be derived from a human. In other cases, the cells may be derived from a non-human animal, including, but not limited to, a non-human primate, a livestock animal, a domestic pet (e.g., a dog, a cat, a sheep, a cow, a horse), or a laboratory animal. For example, a non-human animal can be an ape (e.g., a chimpanzee, a baboon, a gorilla, or an orangutan), an old world monkey (e.g., a rhesus monkey), a new world monkey, a dog, a cat, a bison, a camel, a cow, a deer, a pig, a donkey, a horse, a mule, a lama, a sheep, a goat, a buffalo, a reindeer, a yak, a mouse, a rat, a rabbit, a whale, or any other non-human animal. The encapsulated cells may be from the subject (e.g., autologous cells), from another donor (e.g., allogeneic cells) or from other species (e.g., xenogeneic cells). In a non-limiting example, the cells may be xenogeneic islets or islet cells (e.g., from a pig) that are transplanted into a human subject.

The cells can be introduced into the lumen of the device (e.g., the encapsulation device) and the device may be immediately (within a day) implanted into a subject. Alternatively, the cells may be introduced into the lumen of the device and the device containing the cells encapsulated therein can be cultured for a period of time (e.g., greater than one day) to allow for the cells to proliferate and/or embed into the scaffold of the device, prior to implantation. In some cases, the cells can be cultured for up to 12 hours, up to 1 day, up to 2 days, up to 3 days, up to 4 days, up to 5 days, up to 6 days, up to 7 days, or greater than 7 days, before the device is implanted into the subject.

In some cases, the number of cells introduced into the lumen of the device (e.g., the encapsulation device) is at least 100 cells, at least 200 cells, at least 300 cells, at least 400 cells, at least 500 cells, at least 1.000 cells, at least 2.000 cells, at least 3.000 cells, at least 4.000 cells, at least 5.000 cells, at least 10,000 cells, at least 20.000 cells, at least 30,000 cells, at least 40,000 cells, at least 50.000 cells, at least 60.000 cells, at least 70.000 cells, at least 80.000 cells, at least 90.000 cells, at least 100.000 cells, at least 200.000 cells, at least 300.000 cells, at least 400.000 cells, at least 500.000 cells, at least 600.000 cells, at least 700.000 cells, at least 800.000 cells, at least 900.000 cells, at least 1 million, at least 5 million, at least 10 million, at least 50 million, at least 100 million, at least 200 million, at least 300 million, at least 400 million, at least 500 million, at least 600 million, at least 700 million, at least 800 million, at least 900 million, or at least 1.000 million cells. The number of cells introduced into the lumen of the device may vary and may be determined empirically.

In some embodiments, the cells introduced into the lumen of the device (e.g., the encapsulation device) may be modified. Modifications can include genetic modifications or epigenetic modifications. Genetic modifications can include insertions, deletions, mutations, or inversions. Modifications can be introduced into the cells through gene editing, for example, through the use of a CRISPR/Cas gene editing system. The cells can be modified to alter (e.g., increase, decrease, or eliminate) expression of a protein of interest. The cells can be modified to increase expression of a protein of interest. Genetic modification to alter expression of the protein of interest can comprise modifications to regulatory regions involved in the expression of the protein of interest. Genetic modifications may also comprise virus directed editing. Regulatory regions can include, but are not limited to promoters, transcription factors, enhancers, silencers. 5′UTR, and 3′UTR. The cells can be modified to produce a structural or functional variant of a protein of interest. The cells introduced into the lumen can be recombinant cells.

In some aspects, the cells may be therapeutic cells, for example, cells which produce and release or secrete therapeutic molecules that provide a therapeutic benefit to the biological subject. The device (e.g., the encapsulation device) may be configured such that therapeutic molecules may be released from the device to e.g., effectuate a therapeutic response in the biological subject. The therapeutic molecule can be a protein. In some cases, the protein can be a naturally-produced protein. In some cases, the protein can be a recombinant protein. The protein can be a hormone or an enzyme. The protein can be insulin or an analog thereof, erythropoietin, a coagulation modulator (e.g., coagulant or anticoagulant), a thrombolytic enzyme, an alpha-galactosidase A, deoxyribonuclease I, glucocerebrosidase, iduronidase. N-acetylgalactosamine-4-sulfatase, growth hormone or an analog thereof, a gonadotropin, calcitonin, glucagon, an interferon, an interleukin, thrombopoietin, estrogen, testosterone, or a granulocyte colony-stimulating factor. The coagulant can be Factor-VII A. Factor-VIII, or Factor-IX. The gonadotrophin can be a follitropin, a choriogonadotropin, follicle-stimulating hormone, or luteinizing hormone. The interferon can be an interferon-alpha, an interferon-beta, or a interferon-gamma. The interleukin can be IL-1, IL-2, IL-3, IL-4, IL-10, IL-11, IL-13, IL-15, IL-17, or IL-18. In some cases, the therapeutic molecule is an antibody. In one non-limiting example, the cells are islets or islet cells, and the therapeutic molecule is insulin (e.g., to treat a diabetic subject). The type of cell to be used in the device generally is determined based on the desired therapeutic molecule and the therapeutic response desired in the subject.

In some aspects, the devices (e.g., the encapsulation device) provided herein may be configured to be implanted into a subject. In some cases, the device may be used to transplant or transfer cells into a subject. In some cases, the devices may be implanted into a subject by subcutaneous implantation, intramuscular implantation, subdermal implantation, intraperitoneal implantation, retroperitoneal implantation, omentum implantation, implantation into the brain, subfacial implantation, implantation into the bone, ocular implantation, implantation into an organ, implantation adjacent to an organ (e.g., liver, etc.). The site of implantation may be selected based on the diseased/injured tissue that requires treatment. For treatment of a disease such as diabetes mellitus (DM), the device may be placed in a clinically convenient site such as the subcutaneous space or the omentum.

In certain embodiments, the device may be sealed entirely along the edges of the device thereby forming a completely enclosed internal space or lumen. In other embodiments, the device may be open at one or more locations at an edge of the device allowing access to the internal space or lumen of the device. When the device includes two openings into the lumen of the device, the two openings may be located opposite each other, such as, on opposite sides of the planar device. In some cases, a device of the disclosure may not include a support or frame. For example, the first polymer layer and the second polymer layer may be laid on top of one another and sealed along the periphery of the layers. In some cases, a device of the disclosure may have a support or frame. In such embodiments, the device may be flexible or have a flexible structure such that it can be rolled up. In some cases, the device may be folded or rolled up prior to implantation, and then unfolded or unrolled after implantation.

In some aspects, the cells may be loaded into the lumen of the device prior to implantation into a subject. In some cases, after the cells have been loaded into the lumen of the device, the cells may embed within the macroporous scaffold of the second polymer layer. In other aspects, the device (without any cells loaded therein) may be implanted into the subject. The device may then later be loaded with cells, after the device has been implanted for a period of time. For example, the device (e.g., the encapsulation device) may be implanted into a subject at least 1 day, at least 2 days, at least 3 days, at least 4 days, at least 5 days, at least 6 days, at least 1 week, at least 1.5 weeks, at least 2 weeks, at least 2.5 weeks, at least 3 weeks, at least 3.5 weeks, or at least 4 weeks prior to loading the device with cells. In some aspects, the device may further comprise a port for loading cells into the device. In some cases, the port may be a tubing for introducing cells into the lumen of the device. In certain embodiments, the tubing may be affixed to the device by sealing the first and second layers, at the opening, to the exterior wall of the tubing. In certain embodiments, a first end of the tubing may be placed at the opening and positioned in the lumen such that a minimal volume of the lumen is taken up by the tubing while allowing loading of fluids into the lumen of the device. The second end of the tubing, which is distal to the first end, may be used as a port for introducing fluids into the lumen via the tubing. The length of the tubing may be selected based on a number of factors. For example, a shorter tubing may be used when loading a population of cells into the lumen of the device ex vivo, for example, prior to placement of the device into a subject. A longer tubing may be used if the tubing is to be used for introducing fluids into and/or removing fluids from the lumen of the device after placement of the device into a subject. In some cases, the port may be accessible after the device has been implanted into the subject, such that the device may be pre-implanted without any cells contained within the lumen, and the lumen may be loaded with cells at some period of time after implantation. In some aspects, the device (e.g., the encapsulation device) may be pre-loaded with support cells or factors (e.g., growth factors) prior to introduction of the therapeutic cells.

The devices herein may be designed such that therapeutic molecules (e.g., such as those produced by and secreted from therapeutic cells) can diffuse out of the lumen or internal compartment of the device. Additionally, the devices may allow molecules present outside of the device to permeate into the lumen or internal compartment of the device. In some cases, the device may allow for oxygen to diffuse into and/or out of the device. In some aspects, the devices may promote the growth of blood vessels into or around the implantation site of the device (e.g., promote vascularization of the device).

When an implantable device containing cells has been isolated from the immune system by encasing it in a cell-impermeable layer, the implantable device may often stimulate a local inflammatory response, called the foreign body response (FBR) that has long been recognized as limiting the function of implanted devices that require solute transport. FBR has been well described in the literature and is composed of three main layers. The innermost FBR layer, adjacent to an implanted device is composed of macrophages and foreign body giant cells. These cells form a monolayer of closely opposed cells over the surface of an implanted device. The intermediate FBR layer, lying distal to the first layer with respect to the device, is a wide zone (30-100 microns) composed primarily of fibroblasts and fibrous matrix. The outermost FBR layer is loose connective granular tissue containing new blood vessels. Upon induction of a FBR, an implanted device may be isolated from the in vivo environment limiting the exchange of molecules with the implanted device, limiting the utility of the implanted device as well as, leading to the death of any cells provided within the implanted device (e.g., the encapsulation device). In some cases, a device of the disclosure may not provoke a foreign body response, may not provoke a substantial foreign body response, or may provoke a limited foreign body response, such that the cells encapsulated within the device remain viable for a period of time. In some cases, a foreign body response may be evidenced by fibrosis around the implanted device. In some cases, a device of the disclosure may not provoke fibrosis, may not provoke substantial fibrosis, or may provoke limited fibrosis, around the implanted device.

In some aspects, the devices (e.g., the encapsulation device) disclosed herein may support viability of cells present in the lumen of the device upon transplantation into a subject, for at least one week, two weeks, three weeks, four weeks, one month, at least 2 months, at least 3 months, at least 4 months, at least 5 months, at least 6 months, at least 7 months, at least 8 months, at least 9 months, at least 10 months, at least 11 months, at least 12 months, at least 2 years, at least 3 years, at least 4 years, at least 5 years, or longer.

In some aspects, the devices (e.g., the encapsulation device) disclosed herein may be substantially impermeable to cells present outside the device such that cells present outside the device do not enter the device. For example, in some cases, the device is substantially impermeable to immune cells such that immune cells present outside the device may not enter the lumen of the device. In some aspects, the devices disclosed herein may be substantially impermeable to molecules present outside the device such that said molecules do not enter the device. For example, in some cases, the device is substantially impermeable to immunoglobulins such that the concentration of any immunoglobulins that gain access to the lumen of the device is below the level needed for an immune response against the cells present inside the lumen of the device. In another example, the devices of the present disclosure may substantially limit cytokines from entering the lumen of the device such that the concentration of cytokines that gain access to the lumen of the device is below the level required for an immune response against the cells present inside the lumen of the device. In some aspects, the devices may be configured to promote nutrient exchange with the encapsulated cells and/or diffusion of metabolic waste products out of the device. In some cases, the devices provided herein may be configured to permit the passage of small molecules (including therapeutic molecules), but to substantially inhibit passage of large molecules.

In some aspects, the exterior surface of the device (e.g., the encapsulation device) may be modified by coating with one or more agents that improve a function of the device. For example, molecules that promote vascularization of the device or inhibit immune or inflammatory responses to the device may be disposed on the exterior of the device. In some cases, agents may be disposed within the device (e.g., loaded inside the lumen of the device, or coated on an interior surface of the device). In some cases, the agents may diffuse out of the lumen of the device and act on an external environment. Additionally or alternatively, the agents inside the lumen of the device may act on cells disposed within the lumen of the device (e.g., maturation factors that act on partially mature stem cells). Such molecules or agents may include, but are not limited, to VEGF (vascular endothelial growth factor), PDGF (platelet-derived growth factor). FGF-1 (fibroblast growth factor), angiopoietin MCP-1, αᵥβ₃, αᵥβ₅. CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like.

Further provided herein are methods for using the subject devices (e.g., the encapsulation device). In one aspect, a device of the disclosure may be implanted into a subject. In some cases, the device may be implanted into the subject with cells pre-loaded into the lumen of the device. In other cases, the device may be implanted into the subject without cells pre-loaded into the lumen of the device. In such cases, the device may, after a period of time, be loaded with cells (e.g., by using a pre-implantation port or tubing of the device). In some cases, the methods may involve using the subject devices for transplanting cells that produce and secrete therapeutic molecules into a subject. In some cases, the therapeutic molecules may diffuse out of the device and into an environment of the subject, such that the therapeutic molecule has a therapeutic effect on the subject.

In some cases, the methods may be used to treat a disease or disorder in the subject. The disease or disorder may be a disease or disorder characterized by lack of functional cells or by deficient production of a protein. In some case, the disease or disorder can be, without limitation, diabetes, anemia, a clotting disorder (e.g., hemophilia), cardiovascular disease. Fabry disease. Gaucher disease, mucopolysaccharidosis, growth hormone deficiency, infertility, osteoporosis, hypoglycemia. Parkinson's disease, or muscular dystrophy.

In a non-limiting example, the methods may be used to transplant islets or islet cells into a subject, wherein the islets or islet cells produce and secrete insulin. In such cases, the methods may be used to treat a subject having or suspected of having diabetes. The diabetes can be type 1 diabetes. The device may include pancreatic islet cells or may include stem cells that are capable of differentiating into pancreatic islet cells. In certain embodiments, pluripotent stem cells (PSCs) may be differentiated into pancreatic islet cells inside the device and then the device containing the differentiated pancreatic islet cells is placed in the subject (e.g., in the omentum, adjacent to pancreas or liver). In some case, the device may include PSCs and the device may be implanted adjacent the pancreas or liver of the subject.

Encapsulation devices may be used to transplant a variety of different cell types into a biological subject. Encapsulation devices may further be used to transplant an assortment or a combination of different cell types into a biological subject. Non-limiting examples of cell types that may be used include: thyroid cells, parathyroid cells, bone marrow cells, mesenchymal stem cells, stromal cells, pluripotent stem cells, induced pluripotent stem cells, embryonic stem cells, blood vessel cells, cells derived from adipose tissue, cells derived from bone marrow, intestinal cells or cells derived therefrom, islets or islet cells. Sertoli cells, beta cells, progenitors of islet cells, progenitors of beta cells, peripheral blood progenitor cells, stem cells or derivatives thereof isolated from adult tissue, retinal progenitor derivative cells, cardiac progenitor derivative cells, osteoprogenitor cells, neuronal progenitor cells, genetically transformed cells, or any combination thereof. Derivative cells may include isolated subsets of primary tissue cells in an unaltered state or modified through culture conditions or introduction of materials intracellularly.

In some cases, the cells are therapeutic cells, which may release molecules that provide a therapeutic benefit to the biological subject. The encapsulation devices may be designed such that the therapeutic molecules released from the therapeutic cells can diffuse out of the lumen or internal compartment of the encapsulation device. In some cases, the encapsulation devices are composed of a permeable material. In some cases, the encapsulation devices are composed of a semi-permeable material. Additionally, the encapsulation devices may allow molecules present outside of the encapsulation device to permeate into the lumen or internal compartment of the encapsulation device. In some cases, the encapsulation device may allow for oxygen to diffuse into the encapsulation device. In some cases, the encapsulation device may allow for oxygen to diffuse out of the encapsulation device. In some cases, the encapsulation device may allow for oxygen to diffuse into and out of the encapsulation device.

### Oxygenation and Vascularization

In one aspect, a method is provided wherein cells may be pre-conditioned to hypoxia (e.g., low-oxygen conditions) prior to being transplanted into a host (e.g., a human). In one instance, pre-conditioning of cells to hypoxia may involve exposing the cells to low oxygen conditions (e.g., 0.1%-5% oxygen), by, e.g., culturing cells in low oxygen environments. For example, cells may be exposed to 0.1% oxygen, 0.2% oxygen, 0.3% oxygen, 0.4% oxygen, 0.5% oxygen, 0.6% oxygen, 0.7% oxygen, 0.8% oxygen, 0.9% oxygen, 1.0% oxygen, 1.1% oxygen, 1.2% oxygen, 1.3% oxygen, 1.4% oxygen, 1.5% oxygen, 1.6% oxygen, 1.7% oxygen, 1.8% oxygen, 1.9% oxygen, 2.0% oxygen, 2.1% oxygen, 2.2% oxygen, 2.3% oxygen, 2.4% oxygen, 2.5% oxygen, 2.6% oxygen, 2.7% oxygen, 2.8% oxygen, 2.9% oxygen, 3.0% oxygen. 3.1% oxygen, 3.2% oxygen, 3.3% oxygen, 3.4% oxygen, 3.5% oxygen, 3.6% oxygen, 3.7% oxygen, 3.8% oxygen, 3.9% oxygen, 4.0% oxygen, 4.1% oxygen, 4.2% oxygen, 4.3% oxygen, 4.4% oxygen, 4.5% oxygen, 4.6% oxygen, 4.7% oxygen, 4.8% oxygen, 4.9% oxygen, or 5.0% oxygen. The cells may be exposed to greater than or less than 5.0% oxygen. The cells may be exposed to low oxygen conditions for a period of time. The cells may be exposed to low oxygen conditions for a period of time prior to being transplanted into a host (e.g., a human). In some examples, the cells may be pre-conditioned to hypoxic conditions prior to being loaded into an encapsulation device. In other examples, the cells may be loaded into an encapsulation device as described here, and then may be pre-conditioned to hypoxic conditions, such as by incubating the encapsulation device in hypoxic conditions. The encapsulation device comprising the pre-conditioned cells may then be implanted into a host.

In some cases, the cells may be pre-conditioned by exposure to low oxygen conditions for at least 10 minutes to 60 minutes prior to transplantation into a host. For example, the cells may be exposed to low oxygen conditions for at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, or at least 60 minutes prior to transplantation into the host. In other cases, the cells may be pre-conditioned by exposure to low oxygen conditions for at least 1 hour to 24 hours prior to transplantation into the host. For example, cells may be exposed to low oxygen conditions for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours prior to transplantation into a host. In other cases, the cells may be pre-conditioned by exposure to low oxygen conditions for at least 1-day to 7 days prior to transplantation into the host. For example, cells may be exposed to low oxygen conditions for at least 1 day, at least 2 days, at least 3 days, at least 4 days, at least 5 days, at least 6 days, or at least 7 days prior to transplantation into a host. In yet other cases, cells may be pre-conditioned by exposure to low oxygen conditions for at least 1 week to 4 weeks prior to transplantation into the host. For example, cells may be exposed to low oxygen conditions for at least 1 week, at least 2 weeks, at least 3 weeks, or at least 4 weeks prior to transplantation into the host. The cells may be exposed to low oxygen conditions for a sustained period of time. The cells may be exposed to low oxygen conditions for a sustained period of time without interruption. Additionally or alternatively, the cells may be exposed to low oxygen conditions and high oxygen conditions in a cyclical fashion. For example, cells may be exposed to low oxygen conditions (e.g., 0.1%-5% oxygen) for a period of time. Subsequently, the cells may then be exposed to high oxygen conditions (e.g., 19-21% oxygen) for a period of time, and then subsequently may be exposed again to low oxygen conditions (e.g., 0.1%-5% oxygen) for a period of time, and so forth. The cells may be exposed to high oxygen conditions. For example, the cells may be exposed to 19% oxygen. 19.3% oxygen. 19.6% oxygen. 19.9% oxygen. 20% oxygen. 20.3% oxygen. 20.6% oxygen. 20.9% oxygen, or 21% oxygen. The cells may be exposed to greater than or less than 21% oxygen.

In another aspect, a method is provided for pre-vascularization of an implantation site prior to implantation of an encapsulation device comprising therapeutic cells. In one example, an implantation site is selected in a subject. In one example, the implantation site is subcutaneous. The site of implantation may be selected based on the diseased/injured tissue that requires treatment. For treatment of a disease such as diabetes mellitus (DM), the device may be placed in a clinically convenient site such as the subcutaneous space or the omentum. In some embodiments, factors may be added to the pre-vascularized implantation site. In some embodiments, the factor may comprise oxygen. In other embodiments, the factor may not comprise oxygen. In some embodiments, one factor may be added. In some embodiments, more than one, more than two, more than three, more than four, more than five, more than six, more than seven, more than eight, more than nine, or more than ten factors may be added to the pre-vascularized implantation site. The factors may also include, but are not limited to: anti-lymphocyte function-associated antigen 1 (anti-LFA1) and analogs thereof (e.g., efalizumab), vascular endothelial growth factor (VEGF), platelet-derived growth factor (PDGF), fibroblast growth factor (FGF-1), angiopoietin, monocyte chemoattractant protein 1 (MCP-1), integrin alpha-v beta-3, integrin alpha-v beta-5, platelet endothelial cell adhesion molecule-1 (PECAM-1/CD-31), vascular endothelial cadherin (VE-cadherin), ephrin, plasminogen activators (e.g., urokinase (UA), tissue plasminogen activator (tPA), angiogenin. Del-1, acid fibroblast growth factor (aFGF), basic fibroblast growth factor (bFGF), follistatin, granulocyte colony-stimulating factor (G-CSF), hepatocyte growth factor (HGF), leptin, placental growth factor, platelet-derived endothelial growth factor (PD-ECGF), and the like. In some embodiments, the added factors may help the engrafted cells connect to oxygen or a source of oxygen. In other embodiments, the added factors may not help the cells connect to oxygen or a source of oxygen.

The methods described herein may comprise a pre-vascularization device. The prevascularization device may comprise similar composition, shape, and/or size to the encapsulation device. The pre-vascularization device may be implanted into the implantation site after the implantation of the encapsulation device comprising the therapeutic cells. In some cases, the devices may be implanted into a subject by subcutaneous implantation, intramuscular implantation, subdermal implantation, intraperitoneal implantation, retroperitoneal implantation, omentum implantation, implantation into the brain, subfacial implantation, implantation into the bone, ocular implantation, implantation into an organ, implantation adjacent to an organ (e.g., liver, etc.). For example, if the implantation site is subcutaneous, the pre-vascularization device may be implanted under the skin. In some cases, the pre-vascularization device comprises the same materials as the encapsulation device. In some cases, the pre-vascularization device comprises materials that are similar to the materials of the encapsulation device. In a non-limiting example, when the encapsulation device comprising a permeable or semi-permeable membrane is to be used to deliver therapeutic cells to a subject, the pre-vascularization device may comprise an impermeable membrane that comprises the same or similar materials as the encapsulation device.

In one example, the encapsulation device comprises a semi-permeable biocompatible polymer membrane and the pre-vascularization device comprises an impermeable biocompatible polymer membrane. In some cases, the biocompatible polymer is poly-caprolactone (PCL). In some cases, the pre-vascularization device comprises a biodegradable impermeable membrane that is capable of dissolving within the implantation site.

Additionally or alternatively, the pre-vascularization device may be of similar dimensions and/or of similar shape as the encapsulation device. In some cases, the pre-vascularization device and the encapsulation device may have a two-dimensional planar shape. For example, the two-dimensional planar shape may be, without limitation, circular, elliptical, square, or rectangular, or combinations thereof. In some cases, the pre-vascularization device and the encapsulation device may have a three-dimensional shape. For example, the three-dimensional shape may be, without limitation, spherical, conical, cuboidal, cylindrical, triangular, hexagonal, tetrahedrical, pyramidal, or octagonal, or combinations thereof. The pre-vascularization device may be implanted into the implantation site prior to the implantation of the encapsulation device. The pre-vascularization device may be implanted into the implantation site at least an hour, at least 2 hours, at least 3 hours, at least 4 hours, at least 5 hours, at least 6 hours, at least 7 hours, at least 8 hours, or at least 9 hours prior to implantation of the encapsulation device. The pre-vascularization device may be implanted into the implantation site at least a day, at least 2 days, at least 3 days, at least 4 days, at least 5 days, at least 6 days, at least 7 days, at least 8 days, or at least 9 days prior to implantation of the encapsulation device. The pre-vascularization device may be implanted into the implantation site at least a week, at least 2 weeks, at least 3 weeks, at least 4 weeks, at least 5 weeks, at least 6 weeks, at least 7 weeks, at least 8 weeks, or at least 9 weeks prior to implantation of the encapsulation device. In some cases, the pre-vascularization device may promote the growth of blood vessels. In some cases, the pre-vascularization device may promote the growth of blood vessels into the implantation site.

In some aspects, a surface of the pre-vascularization device may be coated with one or more molecules. The surface of the pre-vascularization device may be coated with one or more molecules that promote vascularization of the device. The surface of the pre-vascularization device may be coated with one or more molecules that inhibit an immune or inflammatory response to the device. The surface of the pre-vascularization device may be coated with one or more molecules that promote vascularization of the device or inhibit inflammatory response to the device. Such molecules include, but are not limited to: anti-lymphocyte function-associated antigen 1 (anti-LFA1) and analogs thereof (e.g., efalizumab), vascular endothelial growth factor (VEGF), platelet-derived growth factor (PDGF), fibroblast growth factor (FGF-1), angiopoietin, monocyte chemoattractant protein 1 (MCP-1), integrin alpha-v beta-3, integrin alpha-v beta-5, platelet endothelial cell adhesion molecule-1 (PECAM-1/CD-31), vascular endothelial cadherin (VE-cadherin), ephrin, plasminogen activators (e.g., urokinase (UA), tissue plasminogen activator (tPA), angiogenin. Del-1, acid fibroblast growth factor (aFGF), basic fibroblast growth factor (bFGF), follistatin, granulocyte colony-stimulating factor (G-CSF), hepatocyte growth factor (HGF), leptin, placental growth factor, platelet-derived endothelial growth factor (PD-ECGF), and the like.

The implantation site may be pre-vascularized. After the implantation site has been sufficiently pre-vascularized, the pre-vascularization device may be removed from the implantation site. In some cases, the pre-vascularization device may comprise a biodegradable material. In some cases, the pre-vascularization device may be dissolved or degraded at the implantation site. After removal of the pre-vascularization device, the encapsulation device, which may comprise therapeutic cells and/or molecules, may be implanted into the pre-vascularized implantation site. The pre-vascularization device may promote the growth of blood vessels to or at the site of the implantation, thus pre-vascularizing the transplantation site prior to implantation of the cell-encapsulation device and promoting survival of therapeutic cells contained within the encapsulation device.

In another aspect, a method is provided for pre-vascularization of an encapsulation device. In some instances, an implantation site is selected in a biological subject (e.g., a human). In some cases, an encapsulation device does not comprise cells (e.g., is cell-free). The encapsulation device which does not contain cells may be implanted into the implantation site of the biological subject. In some cases, the cell-free encapsulation device may be implanted into the implantation site at least an hour, at least 2 hours, at least 3 hours, at least 4 hours, at least 5 hours, at least 6 hours, at least 7 hours, at least 8 hours, or at least 9 hours prior to transplantation of therapeutic cells into the biological subject. In some cases, the cell-free encapsulation device may be implanted into the implantation site at least a day, at least 2 days, at least 3 days, at least 4 days, at least 5 days, at least 6 days, at least 7 days, at least 8 days, or at least 9 days prior to transplantation of therapeutic cells into the biological subject. In some cases, the cell-free encapsulation device may be implanted into the implantation site at least a week, at least 2 weeks, at least 3 weeks, at least 4 weeks, at least 5 weeks, at least 6 weeks, at least 7 weeks, at least 8 weeks, or at least 9 weeks prior to transplantation of therapeutic cells into the biological subject. The cell-free encapsulation device may promote the growth of blood vessels to or at the site of implantation. In some cases, after the cell-free encapsulation device is introduced into the implantation site for a period of time, blood vessels may come into contact with the surface of the cell-free encapsulation device and/or the lumen of the cell-free encapsulation device. After the cell-free encapsulation device has become sufficiently vascularized, cells may be introduced into the lumen or internal compartment of the encapsulation device. In a non-limiting example, cells may be introduced into the encapsulation device by way of a remote fill port. Examples of remote fill ports that may be used to introduce cells into an encapsulation device are described in WO2016/028774, which is herein incorporated by reference in its entirety.

In another aspect, methods and devices are provided for introducing oxygen into an implantation site of a subject. In some cases, an oxygenation device may be used to introduce oxygen to the implantation site of a subject. In some instances, the oxygenation device may be a device that may include an oxygen-releasing material. For example, the oxygen-releasing material may be incorporated within the oxygenation device (e.g., encapsulated within a polymer matrix, or absorbed into a structural material of the device, etc.) Non-limiting examples of oxygen-releasing materials include, but are not limited to: sodium percarbonate, calcium peroxide, magnesium peroxide, hydrogen peroxide, and fluorinated compounds. In other instances, the oxygenation device may be an electrolytic oxygen pump. In other instances, the oxygenation device may include chemicals that react to produce oxygen. In other instances, the oxygenation device may be a device that is connected to an oxygen generation pump. In some cases, the oxygen generation pump may be located externally (e.g., outside of the implantation site). In other cases, the oxygen generation pump may be co-implanted with the oxygenation device.

The oxygenation device may be co-implanted with an encapsulation device. In some cases, a cell-free encapsulation device is co-implanted with the oxygenation device, and then cells are later introduced into the encapsulation device. In other cases, an encapsulation device comprising cells may be co-implanted with the oxygenation device.

In another aspect, methods and devices are provided for introducing oxygen into an implantation site of a subject (e.g., a human). In some cases, an oxygenation device is used to introduce oxygen to the implantation site. In some cases, the oxygenation device is co-implanted with an encapsulation device. In some cases, the oxygenation device may be in contact with or in close proximity to the encapsulation device, such that oxygen released from the oxygenation device may penetrate into the encapsulation device. In some cases, the oxygenation device may be rechargeable in situ, such that the oxygenation device may not need to be removed from the subject to recharge the oxygenation device. An oxygenation device of the disclosure may be recharged any number of times and may be rechargeable at least for the life of the transplantation. The oxygenation device may supply sufficient oxygen to the lumen of the encapsulation device, such that the cells contained within the lumen remain viable and functional for a period of time. In some cases, the oxygenation device remains at the implantation site for the life of the transplant. In such situations, the oxygenation device is capable of recharging as frequently as necessary to ensure sufficient oxygen supply to the encapsulation device throughout the life of the transplant. In other cases, the oxygenation device may be removed after engraftment of the encapsulation device. In a non-limiting example where insulin-secreting cells have been transplanted into a subject, engraftment of the encapsulation device may be determined by measuring the Glucose Stimulation Index (GSI) of the insulin-secreting cells within the encapsulation device. In some cases, the oxygenation device may be sheathed in a material. The material may be the same as that which the encapsulation device is composed of. The material may be similar to that which the encapsulation device is composed of. In a non-limiting example, the oxygenation device may be sheathed in a semipermeable PCL membrane.

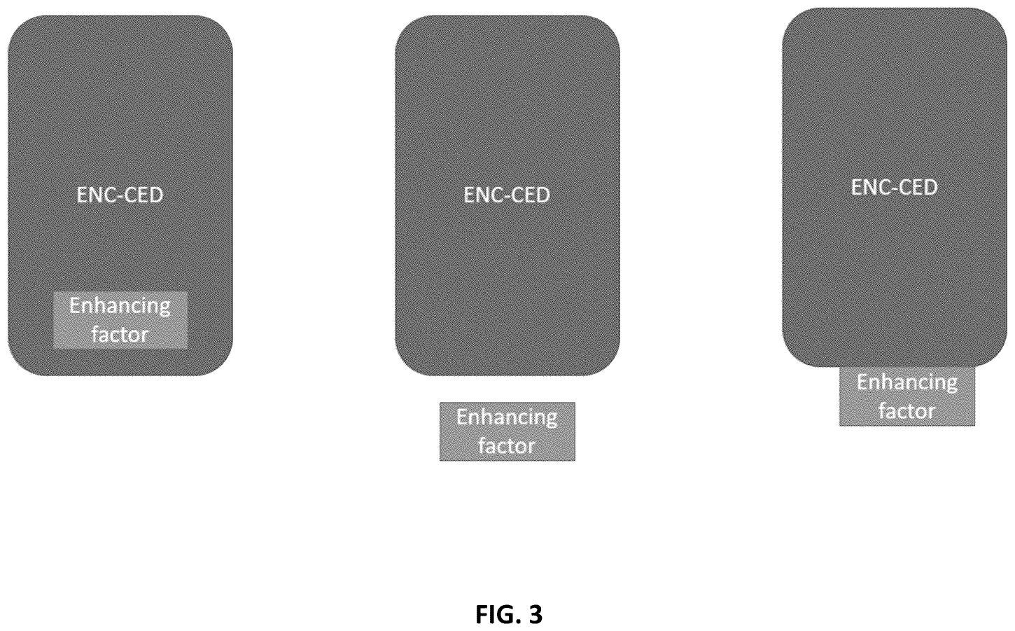

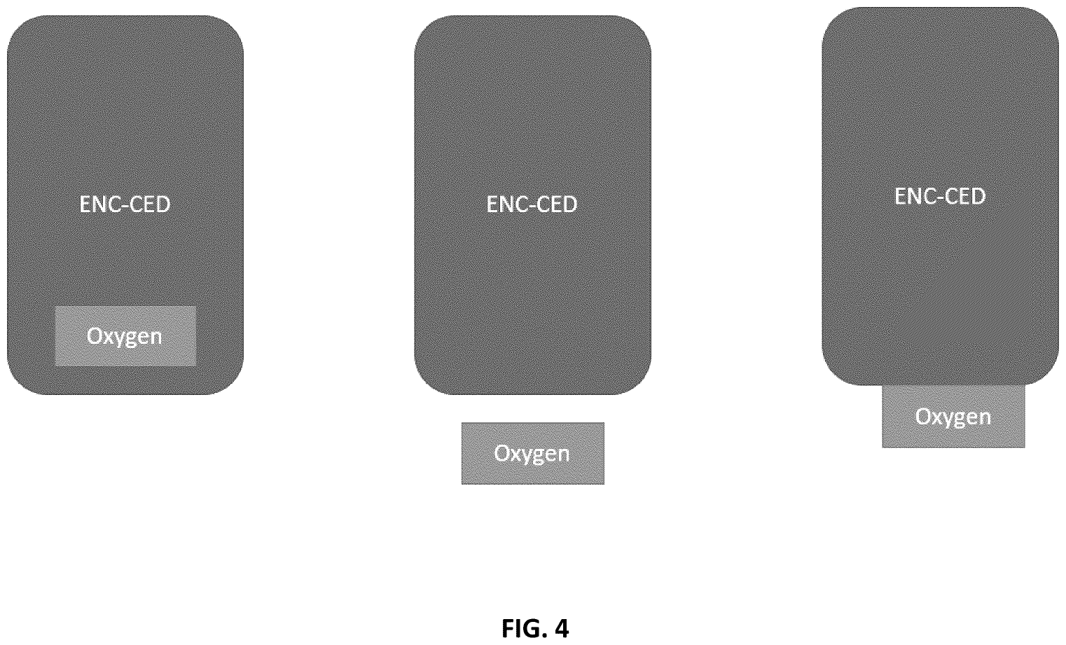

In another aspect, methods and devices are provided for the introduction of chemical supplements to the implantation site. In some cases, the chemical supplements may comprise enhancing factors. Enhancing factors may be delivered inside the device, near the device, or to the device to provide a benefit (FIG. 3). In some cases, the chemical supplements may provide controlled release of oxygen to the implantation site. As shown in FIG. 4, to enhance the device with oxygen, oxygen may be delivered inside the device, near the device, or to the device. Non-limiting examples of chemical supplements that may be used to provide controlled release of oxygen to the implantation site include: hydrogels (e.g., polyethyleneglycole diacrylate (PEGDA)); hemoglobin-based oxygen carriers (HBOCs) including, without limitation, HEMOXCell, PolyHeme®, HemoLink™, and Hemopure®; perfluorodecalin (PFD); perfluorohexyloctane; perfluorates (e.g., copper (I) perfluorate, calcium perfluorate, aluminum perfluorate); perchlorates (e.g., ammonium perchlorate, perchloric acid, potassium perchlorate, sodium perchlorate); and oxygen chelators (e.g., iron, ruthenium, manganese, zinc). Non-limiting examples of mammalian cell types that may be used include, but is not limited to: thyroid cells, parathyroid cells, bone marrow cells, mesenchymal stem cells, stromal cells, pluripotent stem cells, induced pluripotent stem cells, embryonic stem cells, blood vessel cells, cells derived from adipose tissue, cells derived from bone marrow, intestinal cells or cells derived therefrom, islets or islet cells. Sertoli cells, beta cells, progenitors of islet cells, progenitors of beta cells, peripheral blood progenitor cells, stem cells or derivatives thereof isolated from adult tissue, retinal progenitor derivative cells, cardiac progenitor derivative cells, osteoprogenitor cells, neuronal progenitor cells, genetically transformed cells, or any combination thereof. In some embodiments, factors may be delivered from the cell types described herein. In some embodiments, the factors may promote cellular activity. In some embodiments, the factors may promote cellular response. The factors may include, but are not limited to: VEGF (vascular endothelial growth factor), PDGF (platelet-derived growth factor). FGF-1 (fibroblast growth factor), angiopoietin MCP-1, avf33, avf35. CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like. The factors may include vascular inducing factors secreted by the endocrine tissue (e.g., Parathyroid, etc.). The factors may include engraftment supporting factors secreted by the endocrine tissue (e.g., Parathyroid, etc.). The factors may help the encapsulated tissue. The factors may help the graft site. The factors may help the encapsulated tissue and the graft site. The factors may include vascular inducing and/or engraftment supporting factors secreted by endocrine tissues (e.g., Parathyroid, etc.) that may help the encapsulated tissue and/or the graft site. The factors may help secrete or promote. Some examples of factors that may help secrete or promote include, but are not limited to: VEGF (vascular endothelial growth factor). PDGF (platelet-derived growth factor). FGF-1 (fibroblast growth factor), angiopoietin MCP-1, avf33, avf35. CD-31. VE-cadherin, ephrin, plasminogen activators, angiogenin. Del-1, aFGF (acid fibroblast growth factor), vFGF (basic fibroblast growth factor), follistatin. G-CSF (granulocyte colony-stimulating factor). HGF (hepatocyte growth factor). Leptin, placental growth factor. PD-ECGF (platelet-derived endothelial growth factor), and the like.

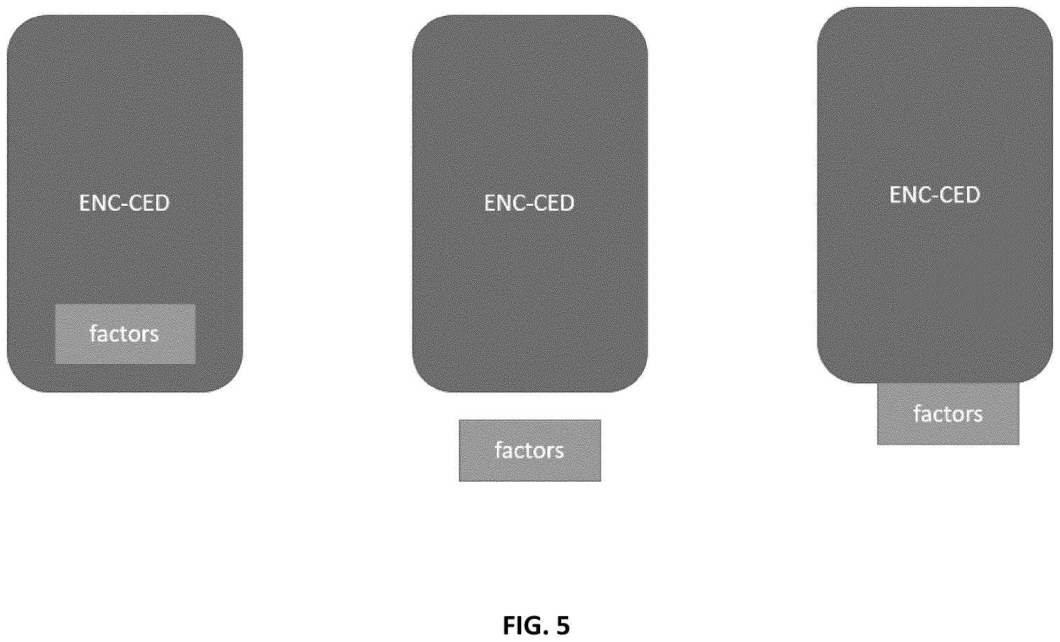

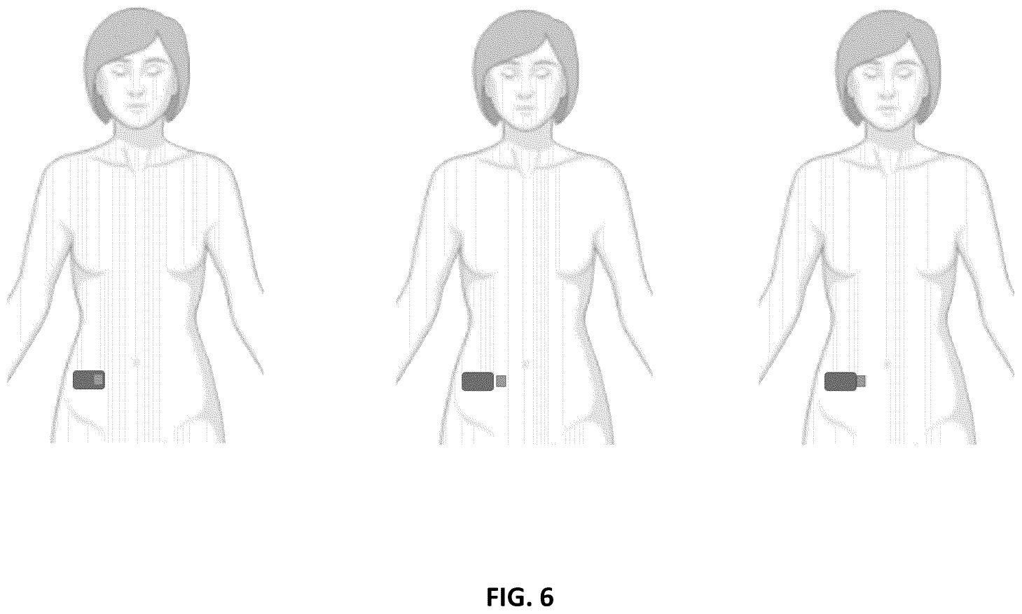

More broadly, the device may be enhanced with factors. Factors may include amino acids, peptides, nucleic acids, sugars, carbohydrates, cytokines, hormones, vitamins, enzymes, growth promotors, proteins, cells, or antioxidants, or combinations thereof. The device may be enhanced with factors by delivering factors inside the device, near the device, or to the device (FIG. 5). The device and the factors may be implanted at an implantation site in a subject (FIG. 6). In some cases, the chemical supplements may be encased within a gel or viscous substance and applied directly to the implantation site. In other cases, chemical supplements may be contained within a porous device that allows for release of oxygen from the device. In one example, chemical supplements may be contained, along with the therapeutic cells, within the lumen of the encapsulation device. In another example, chemical supplements are coated on a surface of the encapsulation device. In yet another example, chemical supplements may be contained on or within a separate device that is co-implanted with the encapsulation device.

In another aspect, methods and devices are provided for introducing supporting cells to the implantation site which may promote more rapid oxygenation of the implant. For example, supporting cells may promote vascularization of the encapsulation device and/or the implantation site. In some cases, the supporting cells may be introduced into the implantation site prior to introduction of the encapsulation device. In other cases, the supporting cells may be contained, along with the therapeutic cells, within the encapsulation device. Non-limiting examples of supporting cells that may be used include, but are not limited to: endothelial cells, oxygen-carrying cells (e.g., blood cells, heme-containing cells or heme-like oxygen chelating cells, etc.), thyroid cells, parathyroid cells, bone marrow cells, mesenchymal stem cells, stromal cells, pluripotent stem cells, induced pluripotent stem cells, embryonic stem cells, blood vessel cells, precursor cells derived from adipose tissue or from bone marrow derived progenitor cells, or from intestinal cells, islets or islet cells. Sertoli cells, beta cells, progenitors of islet cells, progenitors of beta cells, peripheral blood progenitor cells, stem cells or their derivatives isolated from adult tissue, retinal progenitor/derivative cells, cardiac progenitor/derivative cells, osteoprogenitor/derivative cells, neuronal progenitor/derivative cells, genetically transformed cells, or any combination thereof. Derivative cells may include isolated subsets of primary tissue cells in unaltered state or modified through culture conditions or introduction of materials intracellularly.

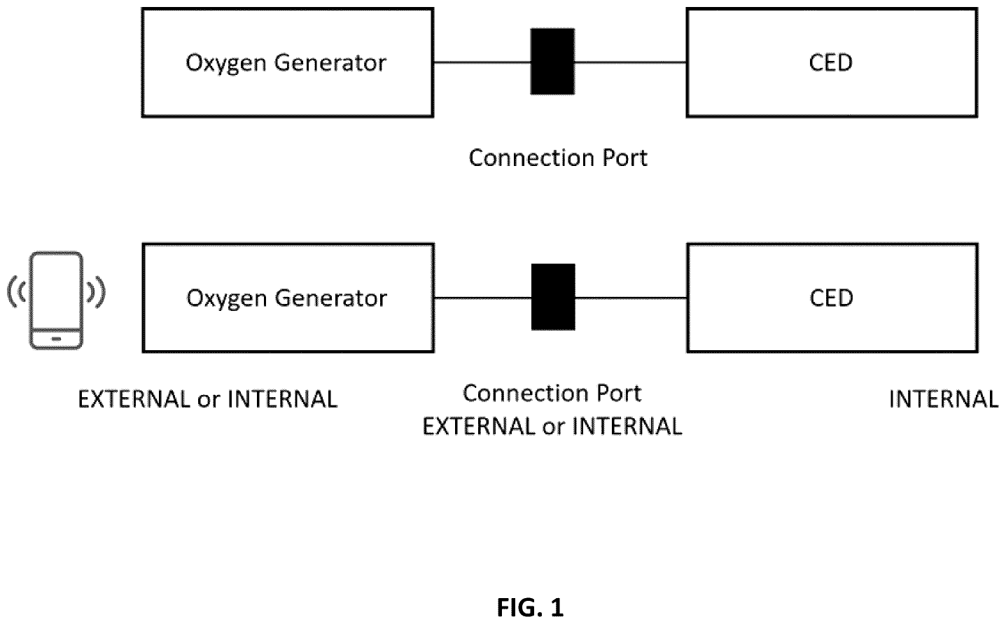

In some embodiments, a system is provided herein. The system may be an oxygenation system. The system may comprise three components: a cell encapsulation device component (e.g., as described herein), an oxygenator component (e.g., a gas/oxygen generator), and a connection port component (FIG. 1). The connection port component may comprise connection accessories.

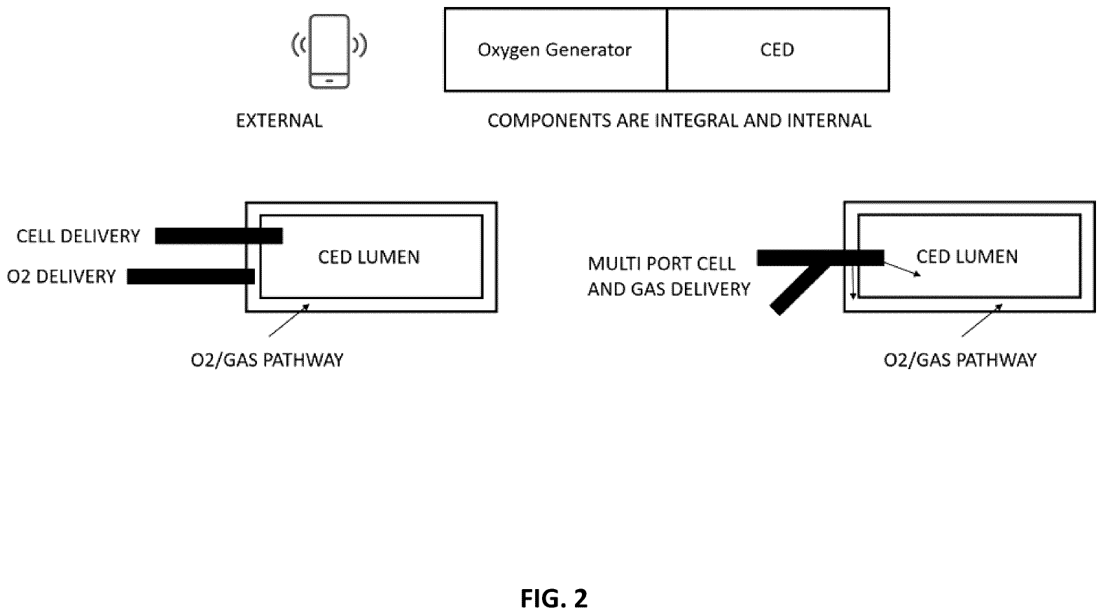

As shown in FIGS. 1 and 2, the cell encapsulation device component may be used within an oxygenation system as described herein. The oxygenation system (e.g., comprising the cell encapsulation device) may be implanted internally. The oxygenation system may be implanted subcutaneously or otherwise in the body of a subject. The cell encapsulation device may comprise a port built into the cell encapsulation device for delivery of the cells (e.g., islets) into the lumen of the cell encapsulation device. The lumen of the cell encapsulation device may comprise cell cargo, a gas/oxygen delivery pathway, and/or a support structure designed to be implanted in the body of a subject. In some embodiments, the gas/oxygen delivery pathway may be a circumferential/perimeter path outlining the cell encapsulation device lumen and support structure. In some embodiments, the gas/oxygen delivery pathway may be layered within the cell encapsulation device construction. In some embodiments, the gas/oxygen delivery pathway may comprise of multiple channels within the cell encapsulation device.

In some cases, the gas/oxygen delivery pathway may be integral to construction of the cell encapsulation device (e.g., the manufacturing processes are performed at the same or similar time to produce a single end device). In some cases, the gas/oxygen pathway may be a separate construction that is subsequently physically connected to the cell encapsulation device. In some cases, the gas/oxygen pathway may not be connected to the cell encapsulation device and may instead be, for example, a donut/toroid surrounding the cell encapsulation device.

The oxygenation system may deliver gas throughout the cell encapsulation device. The oxygenation system may also comprise a return pathway/exhaust. The oxygenation system may comprise multiple return pathways/exhausts. The return pathway/exhaust may be used for waste. The return pathway/exhaust may be used for recycling. The return pathway/exhaust may be used for delivery to multiple cell encapsulation devices that may also be implanted. The return pathway/exhaust may be used for waste, recycling, or delivery, or combinations thereof.

The oxygenation system may be a slow or delayed release system such that the delivery pathway acts like a “sponge”. The oxygen/gas delivery pathway may be saturated with gas/oxygen and slowly diffuse gas/oxygen through to the external cell encapsulation device porous surface for local delivery to tissue. The oxygen/gas delivery pathway may be saturated with gas/oxygen and moderately diffuse gas/oxygen through to the external cell encapsulation device porous surface for local delivery to tissue. The oxygenation system may also deliver oxygen internally to the cell cargo.

The oxygenation system may comprise an outer pouch. The oxygenation system may comprise an inner pouch. The oxygenation system may comprise an outer pouch and an inner pouch such that the configuration of the oxygenation system appears to be a pouch within a pouch as shown in FIG. 2 (e.g., the inner pouch within the outer pouch). In this configuration, the cell encapsulation pouch or device (e.g., the inner pouch) is surrounded by a gas/oxygen delivery pouch (e.g., the outer pouch). The gas/oxygen in the outer pouch may be delivered into the cell encapsulation device (e.g., the inner pouch) directly. The gas/oxygen in the outer pouch may also be diffused through the outer pouch whereby the gas/oxygen may be delivered externally to local tissue located external to the outer pouch.

The oxygenation system may also comprise a gas/oxygen sensor. The gas/oxygen sensor may sense oxygen. The oxygenation may have one or more, two or more, three or more, four or more, five or more, six or more, seven or more, eight or more, nine or more, or ten or more gas/oxygen sensors for sensing oxygen. In some cases, the gas/oxygen sensor may also sense other chemicals. Other chemicals that the gas/oxygen sensor may sense include, but is not limited to, glucose. The one or more sensors may be contained in separate channels or other sealed areas within the cell encapsulation device. The one or more sensors may be contained in the cargo lumen of the cell encapsulation device. The one or more sensors may be contained within the port of the cell encapsulation device. The one or more sensors may be exterior to the cell encapsulation device. The one or more sensors may be within the outer pouch of the inner pouch within the outer pouch configuration.

The oxygenator component in the oxygenation system may be wall sourced powered. The oxygenator component in the oxygenation system may be battery-operated. The oxygenator component in the oxygenation system may be Bluetooth activated. The oxygenator component in the oxygenation system may be Bluetooth monitored. The oxygenator component may comprise a sensor or multiple sensors. The sensor or multiple sensors may activate the oxygenator. Upon activation, the oxygenator may provide or delivery gas. Thus, the oxygenator may provide regulated gas/oxygen delivery. The oxygenator may be implantable. The oxygenator may remain in situ. The oxygenator may be external to the cell encapsulation device. The oxygenator may be wearable with the cell encapsulation device. The oxygenator may be integral to the cell encapsulation device. The cell encapsulation device may be implantable.

The oxygenator (e.g., a gas/oxygen source) may provide gas/oxygen by electrolysis. The oxygenator may provide gas/oxygen by chemical generation. The oxygenator may provide or supply gas/oxygen externally. The oxygenator may deliver gas/oxygen with a carrier media. Examples of carrier media that may be used include hemoglobin and fluorinated microbubbles.

The oxygenation system may comprise a connection port component. The connection port component may provide a connection between the cell encapsulation device and the oxygenator. The connection port component may provide a connection between the cell encapsulation device and the oxygenator when the cell encapsulation device and oxygenator exist as separate components in the oxygenation system. The connection port may comprise accessories. The connection port and connector port accessories may be partially implanted. The connection port and connection port accessories may be fully implanted on a patient. The connection port and connection port accessories may exist entirely externally to the patient. The connection port and connection port accessories may exist partially externally to the patient.

The connection between the oxygen/gas generator and the cell encapsulation device may be sealed connections. The oxygenation system may comprise tubing. The tubing may be between the connection port, the oxygen generator, and the cell encapsulation device. The oxygenation system may comprise valves. The valves may be one-way valves. The one-way valves may be used in the oxygenation system to prevent backflow:

The connection port component in the oxygenation system may provide for cell delivery to the cell encapsulation device cargo lumen. The connection port may comprise a connection. The connection may be used for oxygen/gas delivery. The connection port may comprise a multi-port option. The multi-port option may be used for cell delivery. The multi-port option may be used for oxygen/gas delivery. The multi-port option may be used for both cell delivery and oxygen/gas delivery.

The connection port component of the oxygenation system may comprise accessories. The connection port and accessories may connect the cell encapsulation device and the oxygenator. The connection port and accessories may connect the cell encapsulation device and the oxygenator prior to implantation of the cell encapsulation device. The connection port and accessories may be disconnected from the cell encapsulation device. The connection port and accessories may be reconnected for further oxygen/gas delivery. The connection port and accessories may first connect the cell encapsulation device and the oxygenator prior to implantation of the cell encapsulation device, then be disconnected from the cell encapsulation device, and then at some later time be reconnected for further oxygen/gas delivery.

All components of the oxygenation system (e.g., the cell encapsulation device, the connection port, and the oxygenator) may be placed in a subject. The components of the oxygenation system may be placed in a subject via a pocket created by a surgical incision. The components of the oxygenation system may be placed in a subject via an instrument designed to place the oxygenation system components. The components of the oxygenation system may be sutured in place via suture holes on each component. The components of the oxygenation system may be fit together. The components of the oxygenation system may be friction fit together. In some embodiments, the components of the oxygenation system may be fit together, or friction fit together if the components of the oxygenation system are interlocking. In some embodiments, the components of the oxygenation system may be fit together, or friction fit together if the components of the oxygenation system comprise constraints in size. The components of the oxygenation system may be bonded or welded to each other. In some embodiments, the components of the oxygenation system may be chemically bonded to each other. In some embodiments, the components of the oxygenation system may be thermally bonded to each other. In some embodiments, the components of the oxygenation system may be ultrasonically welded to each other. In some embodiments, the components of the oxygenation system may be chemically bonded, thermally bonded, or ultrasonically welded to each other or combinations thereof.

1-120. (canceled)

121. A device, said device comprising:
(a) an exterior surface, wherein said exterior surface is configured to be in contact with a bodily fluid or a bodily tissue of a subject; and
(b) a lumen, wherein said lumen is configured to encapsulate a plurality of cells, a plurality of therapeutic molecules, or a combination thereof,
wherein said exterior surface of said device or said lumen of said device is modified with a molecule that promotes vascularization of said device, a molecule that inhibits an immune response to said device, a molecule that inhibits an inflammatory response to said device, or a combination thereof.

122. The device of claim 121, wherein said molecule is secreted by a tissue or a population of cells

123. The device of claim 122, wherein said tissue comprises endocrine tissue.

124. The device of claim 122, wherein said population of cells comprise parathyroid cells.

125. The device of claim 121, wherein said molecule is selected from the group consisting of: vascular endothelial growth factor (VEGF), platelet-derived growth factor (PDGF), fibroblast growth factor (FGF-1), angiopoietin, monocyte chemoattractant protein-1 (MCP-1), alphaᵥbeta₃, alphaᵥbeta₅, CD-31, VE-cadherin, ephrin, plasminogen activators, angiogenin, Del-1, acid fibroblast growth factor (aFGF), basic fibroblast growth factor (bFGF), follistatin, granulocyte colony-stimulating factor (G-CSF), hepatocyte growth factor (HGF), leptin, placental growth factor, and platelet-derived endothelial growth factor (PD-ECGF), and any combination thereof.

126. The device of claim 121, wherein said device is configured such that said plurality of therapeutic molecules are capable of diffusing out of said lumen, out of said device, or both.

127. The device of claim 121, wherein said plurality of cells are selected from the group consisting of: bone marrow cells, mesenchymal stem cells, stromal cells, pluripotent stem cells, induced pluripotent stem cells, embryonic stem cells, blood vessel cells, precursor cells derived from adipose tissue, bone marrow derived progenitor cells, intestinal cells, islets, Sertoli cells, beta cells, progenitors of islet cells, progenitors of beta cells, peripheral blood progenitor cells, stem cells isolated from adult tissue, retinal progenitor cells, cardiac progenitor cells, osteoprogenitor cells, neuronal progenitor cells, genetically transformed cells, and any combination thereof.

128. The device of claim 121, wherein said device further comprises a first layer of biocompatible polymer and a second layer of biocompatible polymer.

129. The device of claim 128, wherein said first layer of biocompatible polymer, said second layer of biocompatible polymer, or both, comprise a plurality of nanopores.

130. The device of claim 129, wherein said first layer of biocompatible polymer, said second layer of biocompatible polymer, or both, are selected from the group consisting of: methacrylate polymer, polyethyleneimine, polyethyleneimine-dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapatite, poly(lactic acid), polyhydroxyvalerte and copolymers thereof, polyhydroxybutyrate and copolymers thereof, polycaprolactone, polydiaxanone, polyanhydride, polycyanocrylate, poly(amino acids), poly(orthoesters), polyesters, collagen, gelatin, cellulose polymer, chitosans, alginates, and any combination thereof.

131. The device of claim 130, wherein said first layer of biocompatible polymer and said second layer of biocompatible polymer comprise the polycaprolactone.

132. The device of claim 128, wherein said first layer of biocompatible polymer has a thickness from about 20 μm to about 50 μm.

133. The device of claim 128, wherein said second layer of biocompatible polymer has a thickness from about 1 mm to about 5 mm.

134. The device of claim 121, wherein said device comprises a thickness of less than 40 μm

135. The device of claim 121, wherein said device comprises one or more chemical supplements within said device.

136. The device of claim 135, wherein said one or more chemical supplements are selected from the group consisting of: polyethyleneglycole diacrylate (PEGDA), HEMOXCell, PolyHeme®, HemoLink™, Hemopure®, perfluorodecalin (PFD), copper (I) perfluorate, calcium perfluorate, aluminum perfluorate, ammonium perchlorate, perchloric acid, potassium perchlorate, sodium perchlorate, perfluorohexyloctane, zinc, manganese, ruthenium, and iron

137. A method comprising introducing an encapsulation device to an implantation site of a subject, wherein said encapsulation device comprises: (i) a plurality of therapeutic cells contained within a lumen of said encapsulation device; and (ii) a plurality of supporting cells contained with said lumen of said encapsulation device, wherein said plurality of supporting cells promote oxygenation of said implantation site.

138. The method of claim 137, wherein said plurality of supporting cells promote survival of transplanted cells.

139. The method of claim 137, wherein said plurality of supporting cells promote engraftment.

140. The method of claim 137, wherein said plurality of supporting cells are selected from the group consisting of: endothelial cells, blood cells, heme-containing cells, heme-like oxygen chelating cells, thyroid cells, parathyroid cells, bone marrow cells, mesenchymal stem cells, stromal cells, pluripotent stem cells, induced pluripotent stem cells, embryonic stem cells, blood vessel cells, precursor cells derived from adipose tissue or from bone marrow derived progenitor cells, or from intestinal cells, islets or islet cells, Sertoli cells, beta cells, progenitors of islet cells, progenitors of beta cells, peripheral blood progenitor cells, stem cells or their derivatives isolated from adult tissue, stem cells or their derivatives and cells isolated from adult tissue, stem cells or their derivatives and stem cells isolated from adult tissue, retinal progenitor/derivative cells, cardiac progenitor/derivative cells, osteoprogenitor cells or derivatives thereof, neuronal progenitor cells or derivatives thereof, and any combination thereof.
